(19) United States
(12) Patent Application Publication
(10) Pub. No.: US 2015/0218652 A1
(43) Pub. Date: Aug. 6, 2015

(54) METHODS FOR DIAGNOSIS AND TREATMENT OF CANCER

(71) Applicant: THE REGENTS OF THE UNVERSITY OF COLORADO, a body corporate, Denver, CO (US)

(72) Inventors: Robert C. Doebele, Denver, CO (US); Marileila Varella Garcia, Greenwood Village, CO (US); Anh T. Le, Denver, CO (US)

(21) Appl. No.: 14/423,867

(22) PCT Filed: Aug. 30, 2013
(86) PCT No.: PCT/US2013/057495

§ 371 Date: Feb. 25, 2015

Related U.S. Application Data
(60) Provisional application No. 61/696,002, filed on Aug. 31, 2012.
(60) Provisional application No. 61/827,514, filed on May 24, 2013.

(51) Int. Cl. C12Q 1/68; G01N 33/574

(52) U.S. Cl. CPC C12Q 1/6886; G01N 33/57492; C12Q 2600/106; C12Q 2600/158; G01N 2800/52; G01N 2333/91205

(57) ABSTRACT

Disclosed are markers, methods and assay systems for the identification of patients suspected of having lung cancer and/or cancer patients who are predicted to respond, or not respond to the therapeutic administration of specific chemotherapeutic regimens. Particularly, the invention provides a testing paradigm based on tumor cell samples to select cancer patients who will benefit from chemotherapy including one or more kinase inhibitor(s), as well as a paradigm to select cancer patients who will not benefit from such chemotherapy regimen.

### FIELD OF THE INVENTION

The present invention generally relates to markers, methods and assay kits for the identification of lung cancer patients predicted to respond to specific cancer therapies.

### BACKGROUND OF THE INVENTION

Lung cancer remains a leading cause of mortality in cancer worldwide and is mostly represented by non-small cell lung cancer (NSCLC). NSCLC is increasingly being recognized as a heterogeneous set of diseases based both upon histology as well as molecular characteristics. The identification of these molecular subsets is relevant as there is a growing number of targeted therapies that can effectively inhibit activated oncogenes leading to improved clinical outcomes for patients.

The first important oncogenic fusion in lung cancer was discovered in 2007 by Soda and colleagues. The anaplastic lymphoma kinase gene (ALK) was activated by fusion with the echinoderm microtubule-associated protein-like 4 (EML4). This fusion gene resulted in constitutive activation of the ALK tyrosine kinase domain with activation of downstream signaling pathways and transformed cell growth. Since this discovery, TFG and KIF5B have also been identified as fusion partners for ALK in NSCLC. Crizotinib, a tyrosine kinase inhibitor (TKI) with multiple targets, is currently approved by the US FDA for the treatment of patients with advanced NSCLC proven to be ALK positive (ALK+) and the only FDA approved method for identifying ALK positivity is fluorescence in situ hybridization (FISH) using break-apart (BA) probes specific for regions 5′ and 3′ of the common breakpoint in rearranged ALK. ROS1 is another receptor tyrosine kinase (RTK) recently found to be activated by gene fusions in NSCLC. The first cancer-related genomic rearrangement involving ROS1, an intra-chromosomal deletion on chromosome 6q21 fusing the 5′ region of GOPC to the 3′ region of ROS1, was reported in glioblastoma. In the last five years, 7 different fusions activating ROS1 were identified. Importantly, the ROS1 kinase domain is retained in all of these fusion events and the expressed fusion genes have been reported to be oncogenic. FISH has been a technical platform commonly used to diagnosis these rearrangements, as the nature of the assay allows it to, in theory, detect any all cases in which the ROS1 gene has undergone rearrangement. Recent data also support that lung cancer patients harboring ROS1 gene fusions also respond to crizotinib supporting the clinical utility in identifying these patients.

Four recent studies describe the 3rd gene activated by fusion in lung cancer, RET (Rearranged during Transfection). RET is a well known RTK, with an oncogenic role in papillary and follicular thyroid carcinoma through activation by gene fusions (RET-PTC). Patients whose tumors harbor this fusion respond well to vandetanib, an oral TKI that targets VEGFR, EGFR and RET. A total of 33 patients were reported harboring KIF5B-RET fusions involving 7 different breakpoints. These molecular rearrangements were identified through multiple technologies used to screen more than 2,000 lung adenocarcinomas. In the 3 larger series screened, likely with lower level of pre-selection, frequency of KIF5B-RET fusion ranged from 1% to 2%.

There continues to be a need in the field for identification of further molecular markers, including oncogenic fusion markers, to facilitate more effective detection and treatment of lung cancer.

### SUMMARY OF THE DISCLOSURE

The present invention is based on the discovery of a novel gene fusion comprising the NTRK1 gene that is indicative of lung cancer and also indicative of which patients may respond to cancer therapy comprising therapeutic administration of tyrosine kinase inhibitors.

Accordingly, in one embodiment, the invention comprises a method for determining if a lung cancer patient is predicted to respond to the administration of a chemotherapeutic regimen. The method comprises detecting in a sample of tumor cells from the patient the presence or absence of a marker, wherein the marker comprises a gene fusion comprising a NTRK1-MPRIP gene fusion (NTRK1 encodes the TRKA protein), and wherein the presence or absence of the marker is indicative of whether the cancer patient will respond to the administration of the chemotherapeutic regimen.

In various embodiments, the chemotherapeutic regimen may include administration of one or more of the following: a tyrosine kinase inhibitor, a HSP90 inhibitor (or other chaperone inhibitor), an inhibitor that targets tyrosine kinase downstream signalling cascade, or combinations thereof. Such inhibitors are well known in the art and are commercially available. All such inhibitors are encompassed in the present invention. For instance, in some embodiments, the tyrosine kinase inhibitor may be a TrkA inhibitor, examples of which include, but are not limited to, crizotinib (PF-02340166), ponatinib (AP24534), dovitinib (TK-258), CEP-701, or rebastinib (DCC-2036). Examples of HSP90 inhibitors include, but are not limited to, geldanamycin, herbimycin, 17-AAG, PU24FC1, STA-9090, IPI-504, and AUY-922. Examples of inhibitors that target tyrosine kinase receptor downstream signalling cascade include, without limitation, elumetinib (AZD6244) and MK2206.

In some embodiments, a level of the marker is determined and compared to a standard level or reference range. In some embodiments, the standard level or reference range is determined according to a statistical procedure for risk prediction.

In some embodiments, the presence of the marker may be determined by detecting the presence of a polynucleotide or a polypeptide. In some embodiments, the method may comprise detecting the presence of the polypeptide using a reagent that specifically binds to the polypeptide or a fragment thereof. The reagent may be an antibody, an antibody derivative, or an antibody fragment.

In some embodiments, the presence of the marker may be determined by obtaining RNA from the sample; generating cDNA from the RNA; amplifying the cDNA with primers specific for the marker; and determining from the sequence of the amplified cDNA the presence of absence of the marker in the sample. In some embodiments, the presence of the marker may be determined by Fluorescent In Situ Hybridization (FISH).

The methods of the present invention may further comprise comparing the expression level of the marker in the sample to a control level of the marker selected from the group consisting of: a) a control level of the marker that has been correlated with beneficial response to the administration of a chemotherapeutic regimen including one or more kinase inhibitor(s); and a control level of the marker that has been correlated with lack of beneficial response to the administration of a chemotherapeutic regimen including one or more kinase inhibitor(s); and b) selecting the patient as being predicted to respond to the administration of a chemotherapeutic regimen including one or more kinase inhibitor(s), if the expression level of the marker in the sample is statistically similar to, or greater than, the control level of expression of the marker that has been correlated with sensitivity to the administration of a chemotherapeutic regimen including one or more kinase inhibitor(s), or c) selecting the patient as being predicted to not respond to the administration of a chemotherapeutic regimen including one or more kinase inhibitor(s), if the level of the marker in the sample is statistically less than the control level of the marker that has been correlated with beneficial response to the administration of a chemotherapeutic regimen including one or more kinase inhibitor(s).

In some embodiments, the methods may further comprise comparing the expression level of the marker in the sample to a level of the marker in a second patient predicted to not respond to the administration of a chemotherapeutic regimen including one or more kinase inhibitor(s), and, selecting the patient as being predicted to respond to the administration of a chemotherapeutic regimen including one or more kinase inhibitor(s), if the expression level of the marker in the sample is greater than the level of expression of the marker in the second patient, or, selecting the patient as being predicted to not respond to the administration of a chemotherapeutic regimen including one or more kinase inhibitor(s), if the level of the marker in the sample is less than or equal to the level of expression of the marker in the second patient. In some embodiments the patient is human.

In a further embodiment, the present invention includes an assay system for predicting patient response or outcome to tyrosine kinase anti-cancer therapy comprising a means to detect at least one of: a) the presence of a gene fusion comprising a NTRK1-MPRIP gene fusion; b) the level of expression of a gene transcript encoded by a NTRK1-MPRIP gene fusion; c) the presence of a protein encoded by a NTRK1-MPRIP gene fusion; d) the level of a protein encoded by a NTRK1-MPRIP gene fusion; and, e) the activity of a protein encoded by a NTRK1-MPRIP gene fusion. In some embodiments, the means to detect comprises nucleic acid probes comprising at least 10 to 50 contiguous nucleic acids of NTRK1 gene, or complementary nucleic acid sequences thereof. In some embodiments, the means to detect comprises binding ligands that specifically detect polypeptides encoded by a NTRK1-MPRIP gene fusion. In some embodiments, a surface of the assay system comprises a chip, array, or fluidity card. In some embodiments, the assay system further comprises: a control selected from the group consisting of: information containing a predetermined control level of a gene transcript encoded by a NTRK1-MPRIP gene fusion that has been correlated with response to the administration of a chemotherapeutic regimen including one or more kinase inhibitor(s); and information containing a predetermined control level of a gene transcript encoded by a NTRK1-MPRIP gene fusion that has been correlated with a lack of response to the administration of a chemotherapeutic regimen including one or more kinase inhibitor(s).

In another embodiment, the present invention includes a method of diagnosing a specific type of lung cancer in a subject, comprising detecting in a sample of cells from the subject the presence of a NTRK1-MPRIP gene fusion marker, wherein the presence of the marker is indicative of whether the subject has the specific type of lung cancer. In some embodiments, the presence of the gene fusion marker is detected by RT-PCR or FISH. In some embodiments, the presence of the gene fusion marker is detected by detecting the polypeptide encoded by the gene fusion marker. In some embodiments, the polypeptide is detected by using a reagent that specifically binds to the polypeptide or a fragment thereof.

Other features and advantages of the invention will become apparent to one of skill in the art from the following detailed description.

### BRIEF DESCRIPTION OF THE DRAWINGS

FIG. 1 shows the chromosomal and exon maps of MPRIP gene.

FIG. 2 shows the chromosomal and exon maps of NTRK1 gene.

FIG. 3 shows the arrangement and data confirming NTRK1 gene fusions in lung cancer samples. FIG. 3A is a schematic of genomic rearrangement from tumor samples harboring NTRK1-MPRIP. FIG. 3B shows RT-PCR demonstrating mRNA expression of the novel fusion transcripts. RNA extracted from formalin-fixed paraffin-embedded tumor sample harboring the NTRK1-MPRIP was subject to RT-PCR followed by agarose gel electrophoresis and DNA sequencing. The following abbreviates are used: MPRIP (M), CD74 (C), NTRK1 (N), and exon (ex). Sanger sequencing chromatograms were obtained of RT-PCR product of RNA isolated from tumor samples with NTRK1-MPRIP fusion. SEQ ID NO:1 is the complete cDNA sequence of NTRK1-MPRIP fusion (M21;N14). The cDNA was cloned from a frozen tumor sample from the patient in which this fusion was first identified. Capital letters represent nucleotides contained within the open reading frame.

FIG. 4 shows the RT-PCR primer read sequences for detection of NTRK1-MPRIP gene fusion. FIG. 4A shows the forward read sequence with MPRIP CC3F 1 primer. FIG. 4B shows the reverse read sequence with NTRK1 Y490R1 primer.

FIG. 5 shows the design of the NTRK1-MPRIP fusion FISH probes.

FIG. 6 shows the design of the 5′-3′ NTRK1 Break-Apart FISH probe set aligned against the NTRK1 encoding region of chromosome 1q23.1.

FIGS. 7A and 7B show FISH images obtained from the normal cell line GM09948 using the NTRK1-MPRIP gene fusion probes, providing single clone validation in a normal cell line (Diploid,2N). FIG. 7A: Clone RP11-1038N13 (3′NTRK1) and RP11-125116 (5′MPRIP). FIG. 7B: Clone RP11-1059C21 (3′NTRK1) and Rp11-796J19 (5′MPRIP).

FIGS. 8A and 8B show FISH images obtained from tissue sections that are negative for NTRK1-MPRIP gene fusion using the NTRK1-MPRIP gene fusion probes. FIG. 8A shows specimen S-12-047486 showing low copy number for both genes. FIG. 8B shows specimen S-12-047098, showing high copy number for 3′ NTRK1 and Mid/low copy number for 5′ MPRIP.

FIGS. 9A and 9B show FISH images obtained from tissue sections that are positive for NTRK1-MPRIP gene fusion using the NTRK1-MPRIP gene fusion probes. FIG. 9A showing low copy number for both genes with approximately one red/green fusion (positive pattern) per tumor cell. FIG. 9B showing gene amplification of the fused red/green with mid/low copy number of single reds and single greens per to or cell.

FIG. 10 shows the testing of NTRK1 break-apart FISH probe. FIG. 11a shows cell line GM09948 with a normal karyotype showing metaphase spread and interphase nuclei demonstrating close proximity of the 5′ and 3′ signals indicating an intact NTRK1 gene. FIG. 11b shows KM12 cells which harbor a TPM3-NTRK1 gene fusion showing clear separation of the 5′ and 3′ signals indicating a rearrangement of the NTRK1 gene. FIG. 11c shows a break-apart FISH analysis of NTRK1-MPRIP samples showing clear separation of 5′ and 3′ signals corresponding to the NTRK1 gene. FIG. 11d shows break-apart FISH analysis of a tumor sample without an NTRK1 gene rearrangement showing close approximation of the green/red signals (indicated by arrow).

FIG. 11 shows FISH images obtained from tissue sections that are positive for NTRK1 gene rearrangement using the NTRK1 break-apart probes. Specimen S12-6889 B1 Hybridized with the 5′NTRK1/3′NTRK1 Break Apart probe set. Cells show both the ‘positive’ pattern of split and the ‘normal’ pattern of fused signals.

FIG. 12A and B show immunoblot analyses of cell lysates from 293T cells expressing TRKA. FIG. 12A, Expression of TRKA (with HA tag) or empty vector demonstrates expression of a˜115-120 kD protein detected by an HA-specific antibody (left, Cell Signaling) and a TRKA-specific antibody (right, Santa Cruz, SC-118). FIG. 12B, Immunoprecipation using an HA-specific antibody (Cell Signaling) followed by immunoblot using the same antibody (left) or a phosphotyrosine specific antibody (right, Miilipore, 4G10) following treatment with 1 μM of the indicated inhibitors or DMSO (control) for 5 hours. FIG. 12C shows the expression of NTRK1-MPRIP yields a chimeric protein that is autophosphorylated Immunoblot analysis of 293T cells transiently transfected with empty vector (EV), full length NTRK1 cDNA, NTRK1-MPRIP cDNA compared to tumor cells from a frozen pleural fluid sample or early passage cells in culture (CUTO-3) from the index patient with the NTRK1-MPRIP fusion gene. FIG. 13D is a schematic demonstrating fusion break-point and critical domains of predicted fusion protein products.

FIG. 13A shows immunoblot analyses of downstream signalling of TRKA following treatment with tyrosine kinase inhibitors. SDS-PAGE of 293T cell lysates with expression of TRKA-HA or empty vector in the presence or absence of NGF (10 minutes) and the presence or absence of the indicated tyrosine kinase inhibitors at 1 μM for 5 hours. Membranes were probed with antibodies to TRKA phosphotyrosine 490, 674, and 675 (Cell Signaling), total TRKA (anti-HA, Cell Signaling), AKT phosphoserine 473 (Cell Signaling), total AKT (Cell Signaling), phosphorylated ERK p42/44 (Cell Signaling), total ERK p42/44 (Cell Signaling), and gamma-tubulin (Santa Cruz, SC-8035).

FIG. 13B shows the expression of NTRK1-MPRIP induces activation of downstream MAPK, AKT, and STAT3 pathways. TRKA (NTRK1) fusions are autophosphorylated and activate key downstream signaling pathways. Representative immunoblot analyses (n=3) of cell lysates from Ba/F3 cells expressing RIP-TRKA, the protein product of NTRK1-MPRIP but not its kinase dead (KD) variant display phosphorylation of critical tyrosine residues and activation of pAKT, pERK and pSTAT3 in the absence of IL-3.

FIG. 14 demonstrates that NTRK1 gene fusions support cellular proliferation of Ba/F3 cells in the absence of IL-3. MTS assay of Ba/F3 demonstrates that cells expressing RIP-TRKA, CD74-TRKA, EML4-ALK, or full length TRKA supplemented with NGF proliferate in the absence of IL-3, whereas Ba/F3 cells expressing EV or the kinase dead variant of RIP-TRKA do not proliferate (n=3). Values are mean±SEM.

FIG. 15 demonstrates that NTRK1 fusions support anchorage independent growth. Representative images (n=4) from anchorage independent growth assays of NIH3T3 cells expressing EV, RIP-TRKA-kinase dead (KD), or RIP-TRKA in soft agar.

FIG. 16 shows NTRK1-MPRIP fusion proteins induce tumorigenesis. NIH3T3 cells expressing NTRK1-MPRIP (“RIP-TRKA”), NTRK1-MPRIP kinase dead (“RIP-TRKA-Kinase Dead”), EML4-ALK or Empty Vector were injected into the flanks of nude mice and observed for tumor growth. The number of mice with tumors compared to the total number mice injected are indicated.

FIG. 17 shows RNAi knockdown of NTRK1 inhibits cell proliferation in a cell line harboring TPM3-NTRK1. KM12 cells were analyzed by MTS proliferation assay 96hr after siRNA transfection (n=3). ANOVA analysis followed by Bonferroni's multiple comparison test indicated a significant inhibition of proliferation induced by siRNA 1 (p<0.05). Values represent the mean±SEM. KM 12 cells were transfected with siRNAs targeting NTRK1 and then harvested 48 hr later. Cell lysates were analyzed by immunoblot to detect TRKA, pERK1/2 and ERK1/2.

FIG. 18 shows drug inhibition of activation of TRKA and downstream signaling. Ba/F3 cells expressing NTRK1-MPRIP (RIP-TRKA) or empty vector (EV) were lysed after 5 h of treatment with the indicated doses of drugs (ARRY-470, crizotinib, CEP-701, ARRY-772, or ARRY-523) or DMSO control (C).

FIG. 19 shows that drug treatment inhibits NTRK1 fusion-mediated Ba/F3 cell proliferation and shows the treatment of index patient with crizotinib. Treatment of Ba/F3 cells expressing NTRK1 fusions with TRKA inhibitors inhibits cell proliferation as measured by MTS assay (n=5). FIG. 19(a) Values represent the mean±SEM. Ba/F3 cells expressing NTRK1-MPRIP demonstrate inhibition of proliferation by the pan-TRK inhibitors, ARRY-470, -523, and -772 and the multi-kinase inhibitor, CEP-701, but not the EGFR inhibitor, gefitinib. FIG. 19(b) Crizotinib leads to inhibition of Ba/F3 expressing NTRK1 fusions, similar to Ba/F3 cells expressing ALK or ROS1 fusion constructs. The half maximal inhibitory concentration (IC₅₀) values are listed (nM).

FIG. 20 shows the drug treatment of Ba/F3 cells in the presence of IL-3. Ba/F3 cells expressing empty vector were grown in the presence of IL-3 and treated with a range of doses of ARRY-470, CEP-701, crizotinib, or gefitinib. IC₅₀ values are listed (n=3). Values represent the mean±SEM.

FIG. 21 shows the expression and drug inhibition of NTRK1 fusions in NIH3T3 cells. NIH3T3 cells expressing RIP-TRKA were treated with the indicated doses of drugs for 5 h prior to cell lysis and immunoblot analysis of pTRKA, TRKA, pAKT, AKT, pERK1/2, ERK1/2, pSTAT3, and STAT3 as indicated.

FIG. 22 shows the inhibition of anchorage-independent growth by drugs with TRKA activity. FIG. 22a, NIH3T3 cells expressing empty RIP-TRKA were seeded in triplicate in soft agar and treated with DMSO (control) or 200 nM of ARRY-470, crizotinib, or CEP-701 for 2 weeks (n=4). Representative images are shown. FIG. 22b, The total colony area for each plate was quantified using MetaMorph software and plotted for each condition. Values represent the mean±SEM.

FIG. 23 shows the short term cell culture from index patient showing the NTRK1-MPRIP fusion. Colorado University Thoracic Oncology (CUTO) 3 cells were derived from a pleural effusion from the index patient harboring the NTRK1-MPRIP gene fusion. Left: NTRK1 FISH analysis of CUTO-3 cells showing a positive signal (split green/red signals). Right: Immunoblot analysis of CUTO-3 cells demonstrating inhibition of pTRKA and pERK by the pan-TRK inhibitor, ARRY-470.

FIG. 24 shows drug treatment of KM12 cells. KM12 cells harboring the TPM3-NTRK1 fusion were lysed following 5 h treatment with the indicated doses of inhibitors and subject to immunoblot analysis (n=3).

FIG. 25 shows that drug treatment of KM12 cells inhibits proliferation. Proliferation of KM12 cells treated with the indicated drugs and doses were assayed for cell proliferation by MTS assay. KM12 cells are inhibited by ARRY-470, CEP-701, and crizotinib, but not gefitinib.

FIG. 26 shows that TRKA inhibition results in the accumulation of KM12 cells in G1 phase. KM12 cells were treated with the indicated doses of drugs for 24 hr. Cells were then stained with propidium iodide and analyzed by flow cytometry. ModFit analysis was used to quantify cell cycle profiles (n=3). Values are the mean±SEM.

FIG. 27 shows that drug treatment with TRKA inhibitors induces apoptosis in KM12 cells. FIG. 27A, KM12 cells were treated for 24 h with the indicated drugs and doses, trypsinized, stained with YO-PRO®) and propidium iodide (PI), and analyzed by flow-cytometry. The percent of cells undergoing apoptosis (YO-PRO® positive and PI negative) are plotted (n=4). Values represent the mean±SEM. FIG. 27B TRKA inhibitors induce cleavage of PARP-1. KM12 cells were treated for 24 h with the indicated drugs and doses. Cells were lysed, separated by SDS-PAGE and subject to immunoblot analysis with the indicated antibodies.

FIG. 28 shows histopathology from index patient harboring NTRK1-MPRIP demonstrating lung adenocarcinoma. FIG. 28(a): Needle core biopsy of primary lung left lower lung mass showing adenocarcinoma. FIG. 28(b): Cell block of fine needle aspirate from the same procedure showing tumor cells. FIG. 28(c): TTF-1 immunohistochemistry (IHC) demonstrating strong nuclear staining in tumor cells. FIG. 28(d): Thyrogloblin IHC demonstrating negative staining in tumor cells. Representative images are shown.

FIG. 29 shows the results of treatment when the index patient (NTRK1-MPRIP) consented to treatment with crizotinib 250 mg PO BID (off-protocol, off-label) given lack of other therapeutic options. FIG. 29A: CT scan of the chest before and after 28d of crizotinib and FIG. 29B: serial CA125 tumor marker levels during crizotinib treatment.

FIG. 30 shows the signal configuration “Dot,” which is the typical round and compact signal and comparison with other signals.

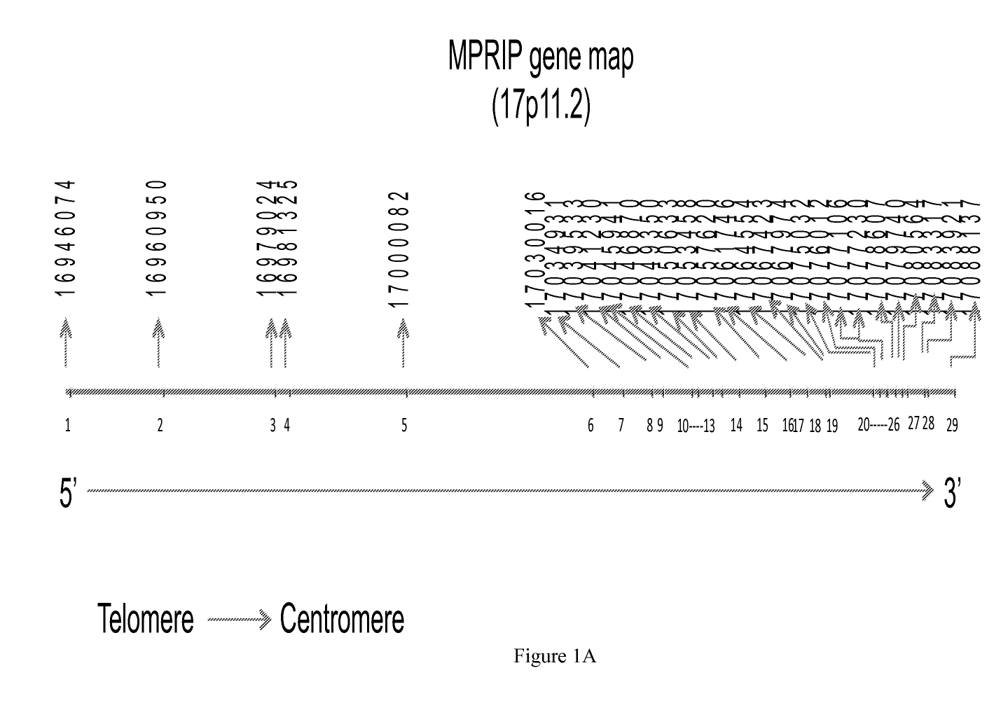

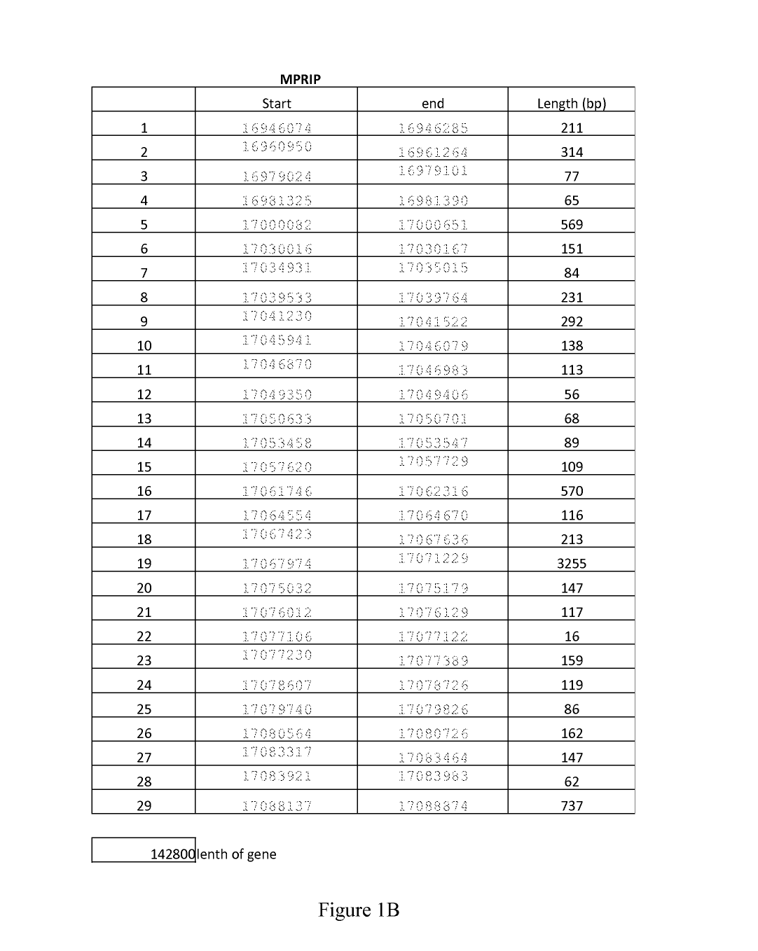

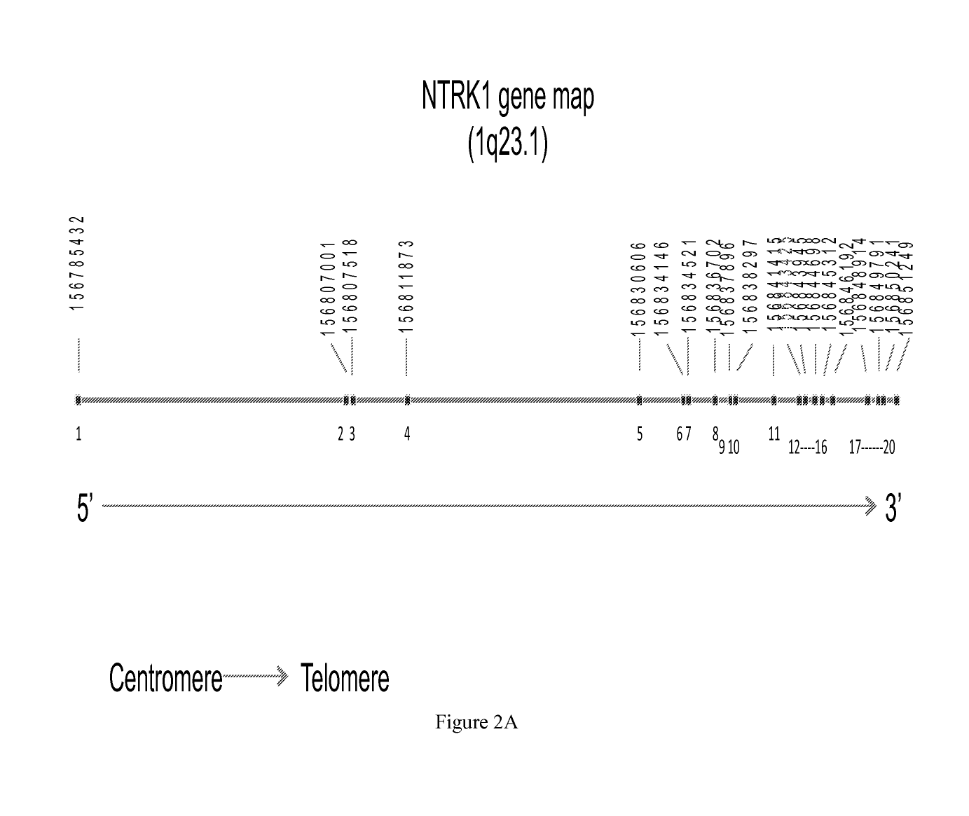

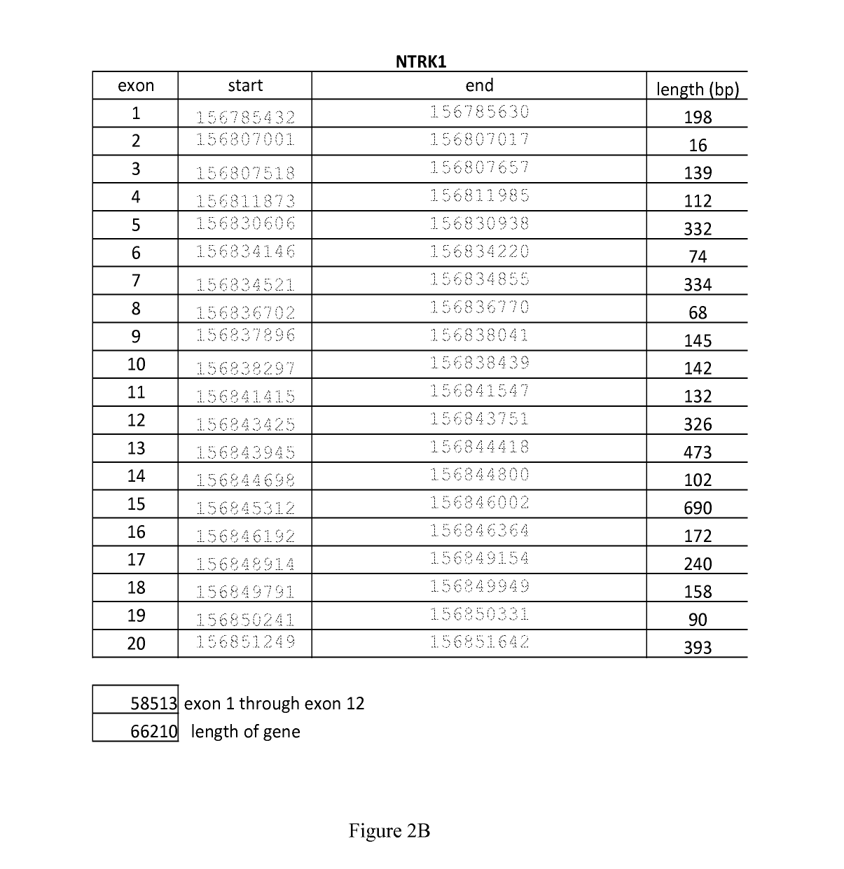

### DETAILED DESCRIPTION OF THE DISCLOSURE

The present inventors have discovered that fusion of the MPRIP and NTRK1 genes is indicative of the presence of a specific type of lung cancer. This gene fusion is also indicative of patient clinical response to treatment with tyrosine kinase inhibitors. This gene fusion, and the levels of the protein encoded by this gene fusion, along with clinical parameters can be used as biological markers to diagnose a specific type of lung cancer and to assess cancer patient response to treatment with tyrosine kinase inhibitors.

According to one definition, a biological marker is “a characteristic that is objectively measured and evaluated as an indicator of normal biologic processes, pathogenic processes, or pharmacological responses to therapeutic interventions.” NIH Marker Definitions Working Group (1998). Biological markers can also include patterns or ensembles of characteristics indicative of particular biological processes (“panel of markers”). The marker measurement can be increased or decreased to indicate a particular biological event or process. In addition, if a marker measurement typically changes in the absence of a particular biological process, a constant measurement can indicate occurrence of that process.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians use the total blood cholesterol as a marker of the risk of developing coronary artery disease, while others use the ratio of total cholesterol to HDL cholesterol.

In the present invention, the markers may be used for diagnostic, prognostic, therapeutic, drug screening and patient stratification purposes (e.g., to group patients into a number of “subsets” for evaluation), as well as other purposes described herein, including evaluation of the effectiveness of a potential cancer therapeutic.

The practice of the invention employs, unless otherwise indicated, conventional methods of analytical biochemistry, microbiology, molecular biology and recombinant DNA generally known techniques within the skill of the art. Such techniques are explained fully in the literature. (See, e.g., Sambrook et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (Glover, ed.); Oligonucleotide Synthesis (Gait, ed., Current Edition); Nucleic Acid Hybridization (Hames & Higgins, eds., Current Edition); Transcription and Translation (Hames & Higgins, eds., Current Edition); CRC Handbook of Parvoviruses, Vol. I & II (Tijessen, ed.); Fundamental Virology, 2nd Edition, Vol. I & II (Fields and Knipe, eds.)).

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers. Unless defined otherwise, all scientific and technical terms are to be understood as having the same meaning as commonly used in the art to which they pertain. For the purposes of the present invention, the following terms are defined below.

As used herein, the term “marker” includes polypeptide markers and polynucleotide markers. For clarity of disclosure, aspects of the invention will be described with respect to “polypeptide markers” and “polynucleotide markers.” However, statements made herein with respect to “polypeptide markers” are intended to apply to other polypeptides of the invention. Likewise, statements made herein with respect to “polynucleotide” markers are intended to apply to other polynucleotides of the invention, respectively. Thus, for example, a polynucleotide described as encoding a “polypeptide marker” is intended to include a polynucleotide that encodes: a polypeptide marker, a polypeptide that has substantial sequence identity to a polypeptide marker, modified polypeptide markers, fragments of a polypeptide marker, precursors of a polypeptide marker and successors of a polypeptide marker, and molecules that comprise a polypeptide marker, homologous polypeptide, a modified polypeptide marker or a fragment, precursor or successor of a polypeptide marker (e.g., a fusion protein).

As used herein, the term “polypeptide” refers to a polymer of amino acid residues that has at least 5 contiguous amino acid residues, e.g., 5, 6, 7, 8, 9, 10, 11 or 12 or more amino acids long, including each integer up to the full length of the polypeptide. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component. Also included are antibodies produced by a subject in response to overexpressed polypeptide markers.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers modified either naturally, or by intervention.

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from a lung cancer subject (or a subject who is suspected of having lung cancer, or is at risk of developing lung cancer) if the level or activity of the marker is higher or lower, respectively, compared to the level of the marker in a sample (or set of samples) obtained from a non-lung cancer subject, or a reference value or range.

The novel gene fusion marker of the present invention was identified as follows: the presence of an oncogene driver gene abnormality was investigated in a non-smoker patient with lung adenocarcinoma. The patient showed no evidence of known mutations, gene amplifications or gene fusions associated with lung cancer. In order to pursue other possible gene targets, genomic DNA from a tumor biopsy sample was analyzed by targeted next generation sequencing, which identified the presence of a novel NTRK1 gene fusion. The gene fusion marker was determined to be a NTRK1-MPRIP gene fusion.

The NTRK1 gene encodes the TRKA receptor tyrosine kinase. The NTRK1 gene has been isolated from a number of species such as human, chimpanzee, dog, cow, mouse, rat, chicken and zebrafish and the sequence determined. All these gene sequences are known to one skilled in the art and are intended to be encompassed in the present invention. Gene fusions involving NTRK1 have previously been reported in papillary thyroid cancer, but have not been reported in lung cancer or other malignancies.

The MPRIP gene encodes the Myosin phosphatase Rho-interacting Protein. The MPRIP gene has been isolated from a number of species such as human, chimpanzee, dog, cow, mouse, rat, chicken, zebrafish and C. elegans and the sequence determined. All these gene sequences are known to one skilled in the art and are intended to be encompassed in the present invention.

This is believed to be the first instance identifying the NTRK1-MPRIP gene fusion in any malignancy. Customized RT-PCR assays, including novel primers, were developed to detect the mRNA transcript of the NTRK1-MPRIP gene fusion. The RT-PCR successfully amplified a small product containing sequences from both MPRIP and NTRK1, confirming expression of a novel gene fusion that included exon 1-21 of MPRIP and exons 14-20 of NTRK1. (See Example 1.) Novel FISH assays were also developed to detect the presence of the NTRK1-MPRIP gene fusion in clinical specimens. (See Examples 2 and 3.)

Additionally, FISH probes that would detect other NTRK1 gene fusions, regardless of the specific 5′ gene fusion partner, were also developed. (See Examples 2 and 4.)

The markers identified herein are of significant biologic interest. Gene fusions involving NTRK1 have previously been reported in papillary thyroid cancer, but have not been reported in lung cancer or other malignancies. Thus, the NTRK1 fusion gene serves as a novel diagnostic marker of cancer. NTRK1 gene encodes the TRKA receptor tyrosine kinase. The presence of the gene fusion was examined in tumor samples obtained from various cancer models, including lung and colorectal cancers, and the sensitivity of the tumor to tyrosine kinase inhibitors was investigated. The objective was to use this gene fusion marker to identify a clinically relevant marker of cancer patient response to tyrosine kinase inhibitor treatment. The methods used are detailed in the Examples section of this disclosure. Several tyrosine kinase inhibitors that are currently in various stages of clinical development including, without limitation, crizotinib, ponatinib, dovitinib, rebastinib, CEP-701, AZD-7451, ARRY-470, ARRY-523, and ARRY-772 as well as other tyrosine kinase inhibitor compounds known in the art that are predicted to inhibit TRKA or oncogenic fusion proteins that contain the TRKA kinase domain, such as NTRK1 fusion proteins. Data presented in Example 5 demonstrates that small molecule tyrosine kinase inhibitors inhibit activated TRKA.

In addition to the discovery of the NTRK1 gene fusion marker that can be used for the diagnosis of, prognosis of, or other evaluation or study of cancer, the marker may also be studied in more detail and/or be used as target for the discovery of other modulators of disease or therapeutic agents.

It is believed that the NTRK1 gene fusion markers, including the NTRK1-MPRIP gene fusion marker, are indicators of cancer patient response to tyrosine kinase inhibitors. Accordingly, in one aspect, the invention provides a marker, the presence or expression level of which is indicative of cancer patient response to tyrosine kinase inhibitors.

In another aspect, the gene fusion markers of the present invention can serve as indicators of cancer patient response to other targeted cancer therapies such as administration of HSP90 inhibitors (or other chaperone inhibitors) or agents that target downstream signalling cascades. Such inhibitors are well known in the art and are commercially available. All such inhibitors are encompassed in the present invention. Examples of HSP90 inhibitors include without limitation geldanamycin, herbimycin, 17-AAG, PU24FC1, STA-9090, IPI-504, and AUY-922. Examples of agents that target downstream signalling cascades include selumetinib (AZD-6244) and MK2206.

The presence of the marker may be detected by detecting a polynucleotide. In one embodiment, the polynucleotide may be a probe that specifically hybridizes with the NTRK1 gene sequences and identifies a chromosomal rearrangement involving the NTRK1 gene. In another embodiment, the polynucleotide may be a primer that specifically binds and amplifies a polynucleotide sequence that is indicative of the presence of the gene fusion involving a NTRK1 gene, including the NTRK1-MPRIP gene fusion marker.

Some variation is inherent in the measurements of the physical and chemical characteristics of the markers of the invention. The magnitude of the variation depends to some extent on the reproducibility of the separation means and the specificity and sensitivity of the detection means used to make the measurement. Preferably, the method and technique used to measure the markers is sensitive and reproducible.

The presence of the gene fusion marker may also be detected by detecting a polynucleotide. Polypeptides corresponding to the NTRK1 gene fusion markers may include a fragment, precursor, successor or modified version of the protein encoded by the NTRK1-gene fusion markers. In another embodiment, the invention includes a molecule that comprises a fragment, precursor, successor or modified polypeptide encoded by the NTRK1-gene fusion markers.

Another embodiment of the present invention relates to an assay system including a plurality of antibodies, or antigen binding fragments thereof, or aptamers for the detection of the expression of the NTRK1 gene fusion markers of the invention. The plurality of antibodies, or antigen binding fragments thereof, or aptamers selectively bind to proteins encoded by the NTRK1 gene fusion markers.

As used herein, the terms “patient,” “subject,” “a subject who has cancer” and “cancer patient” are intended to refer to subjects who have been diagnosed with a cancer or are suspected of having cancer. The terms “non-subject” and “a subject who does not have cancer” are intended to refer to a subject who has not been diagnosed with cancer, or who is cancer-free as a result of surgery to remove one or more tumors. A non-cancer subject may be healthy and have no other disease, or they may have a disease other than cancer. The NTRK1 gene has been found to be conserved in a number of species such as chimpanzee, dog, cow, mouse, rat, chicken, and zebrafish and their sequences are known. In some embodiments, the patient or subject may be a mammal In a preferred embodiment, the patient or subject is human.

Polypeptides encoded by the NTRK1 gene fusion may be isolated by any suitable method known in the art. Native polypeptides encoded by the NTRK1 gene fusion can be purified from natural sources by standard methods known in the art (e.g., chromatography, centrifugation, differential solubility, immunoassay). In one embodiment, the polypeptides may be isolated from a tumor sample. In another embodiment, the polypeptides may be isolated from a sample by contacting the sample with substrate-bound antibodies or aptamers that specifically bind to the marker.

The present invention also includes polynucleotides related to the gene fusion markers of the present invention. In one aspect, the invention provides polynucleotides that comprise the NTRK1 gene fusion markers of the invention. These may be referred to as polynucleotide markers. The polynucleotide markers may be genomic DNA, cDNA, or mRNA transcripts. In another embodiment, the invention provides polynucleotides that have substantial sequence similarity to a polynucleotide that comprises the NTRK1 gene fusion markers or variants thereof, including the NTRK1-gene fusion markers.

In some embodiments, the polypeptides encoded by the NTRK1 gene fusion markers i.e. polypeptide markers may be used as surrogate markers of the NTRK1-MPRIP gene fusion. Thus, for example, if a polypeptide encoded by the NTRK1-MPRIP gene fusion markers is present in cancer patients, the presence or level or activity of the polypeptides may be interrogated (e.g., to identify cancer patients expected to respond to tyrosine kinase inhibitors).

Polynucleotide markers comprising the gene fusion markers may be isolated by any suitable method known in the art. Native polynucleotide markers may be purified from natural sources by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, polynucleotide markers comprising the NTRK1 gene fusion may be synthesized by any suitable chemical or recombinant method known in the art. In one embodiment, for example, the makers can be synthesized using the methods and techniques of organic chemistry. In another embodiment, a polynucleotide marker can be produced by polymerase chain reaction (PCR).

The present invention also encompasses molecules which specifically bind the polypeptide or polynucleotide markers of the present invention. In one aspect, the invention provides molecules that specifically bind to a polypeptide marker or a polynucleotide marker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab') fragments, fusion proteins and any modifications of the foregoing that comprise an antigen recognition site of the required specificity. As used herein, an aptamer is a non-naturally occurring nucleic acid having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In a preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

Certain antibodies that specifically bind polypeptide markers polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind markers of the invention can be used, for example, in methods for detecting protein products encoded by the NTRK1 gene fusion markers of the invention. In one embodiment, antibodies or aptamers against a polypeptide marker or polynucleotide marker of the invention can be used to assay a tissue sample (e.g., a thin cortical slice) for the markers. The antibodies or aptamers can specifically bind to the marker, if any, present in the tissue sections and allow the localization of the marker in the tissue. Similarly, antibodies or aptamers labelled with a radioisotope may be used for in vivo imaging or treatment applications.

The present invention also provides methods of detecting the NTRK1 gene fusion markers of the present invention. The practice of the present invention employs, unless otherwise indicated, conventional methods of analytical biochemistry, microbiology, molecular biology and recombinant DNA techniques within the skill of the art. Such techniques are explained fully in the literature. (See, e.g., Sambrook, J. et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, Vol. I & II (P. Tijessen, ed.); Fundamental Virology, 2nd Edition, Vol. I & II (B. N. Fields and D. M. Knipe, eds.)).

The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a polypeptide or polynucleotide is included in the invention. It is within the ability of one of ordinary skill in the art to determine which method would be most appropriate for measuring a specific marker. Thus, for example, an ELISA assay may be best suited for use in a physician's office while a measurement requiring more sophisticated instrumentation may be best suited for use in a clinical laboratory. Regardless of the method selected, it is important that the measurements be reproducible.

For protein markers, quantification can be based on derivatization in combination with isotopic labelling, referred to as isotope coded affinity tags (“ICAT”). In this and other related methods, a specific amino acid in two samples is differentially and isotopically labelled and subsequently separated from peptide background by solid phase capture, wash and release. The intensities of the molecules from the two sources with different isotopic labels can then be accurately quantified with respect to one another. Quantification can also be based on the isotope dilution method by spiking in an isotopically labelled peptide or protein analogous to those being measured. Furthermore, quantification can also be determined without isotopic standards using the direct intensity of the analyte comparing with another measurement of a standard in a similar matrix.

In addition, one- and two-dimensional gels have been used to separate proteins and quantify gels spots by silver staining, fluorescence or radioactive labelling. These differently stained spots have been detected using mass spectrometry, and identified by tandem mass spectrometry techniques.

A number of the assays discussed above employ a reagent that specifically binds to a NTRK1 gene fusion marker of the invention. Any molecule that is capable of specifically binding to the NTRK1 gene fusion markers of the invention is included within the invention. In some embodiments, the binding molecules are antibodies or antibody fragments. In other embodiments, the binding molecules are non-antibody species, such as aptamers or nucleotide probes.

As described above, the binding molecules may be identified and produced by any method accepted in the art. Methods for identifying and producing antibodies and antibody fragments specific for an analyte are well known.

The markers of the invention also may be detected or measured using a number of chemical derivatization or reaction techniques known in the art. Reagents for use in such techniques are known in the art, and are commercially available for certain classes of target molecules.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labelled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labelled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labelled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2× SSC, followed by additional washes at higher temperatures and lower ionic strength (e.g., at least one wash as about 37° C. in about 0.1×-0.5× SSC, followed by at least one wash at about 68° C. in about 0.1×-0.5× SSC). Other hybridization conditions, and for example, those most useful with nucleic acid arrays, will be known to those of skill in the art.

Using the methods of the present invention, administration of a chemotherapeutic drug or drug combination can be evaluated or re-evaluated in light of the assay results of the present invention. For example, the tyrosine kinase inhibitor drug(s) can be administered differently to different subject populations, depending on the presence of the NTRK1-MPRIP gene fusion markers of the invention in tumor samples from the subjects tested. Results from the different drug regimens can also be compared with each other directly. Alternatively, the assay results may indicate the desirability of one drug regimen over another, or indicate that a specific drug regimen should or should not be administered to a cancer patient. In one preferred embodiment, the finding of the presence of the NTRK1-MPRIP gene fusion markers of the invention is indicative of a good prognosis for response to treatment with chemotherapeutic agents comprising tyrosine kinase inhibitors (“tyrosine kinase inhibitor chemotherapeutic agents”). In another preferred embodiment, the absence of the NTRK1-MPRIP gene fusion markers of the invention in a cancer patient is indicative of a poor prognosis for response to treatment with tyrosine kinase inhibitor chemotherapeutic agents, and may further recommend not administering tyrosine kinase inhibitor chemotherapeutic agent drug regimens. In another aspect, the invention provides a kit for identifying cancer patients predicted to respond or not respond to tyrosine kinase inhibitor drugs, based on the presence or absence of NTRK1-MPRIP gene fusion markers of the disclosure.

The kits of the invention may comprise one or more of the following: an antibody, wherein the antibody specifically binds with a polypeptide marker, a labelled binding partner to the antibody, a solid phase upon which is immobilized the antibody or its binding partner, a polynucleotide probe that can hybridize to a polynucleotide marker, pairs of primers that under appropriate reaction conditions can prime amplification of at least a portion of a gene fusion polynucleotide marker (e.g., by PCR), instructions on how to use the kit, and a label or insert indicating regulatory approval for diagnostic or therapeutic use.

The invention further includes polynucleotide or polypeptide microarrays comprising polypeptides of the invention, polynucleotides of the invention, or molecules, such as antibodies, which specifically bind to the polypeptides or polynucleotides of the present invention. In this aspect of the invention, standard techniques of microarray technology are utilized to assess expression of the polypeptides markers and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Polynucleotide arrays, particularly arrays that bind polypeptides of the invention, also can be used for diagnostic applications, such as for identifying subjects that have a condition characterized by expression of polypeptide markers, e.g., cancer.

The assay systems of the present invention can include a means for detecting in a sample of tumor cells the presence of the NTRK1 gene fusion markers of the invention, and/or a level of expression of the NTRK1-MPRIP gene fusion markers of the invention, and/or a level of protein product of the NTRK1-MPRIP gene fusion markers of the invention.

The assay system preferably also includes one or more controls. The controls may include: (i) a control sample for detecting sensitivity to tyrosine kinase inhibitor chemotherapeutics; (ii) a control sample for detecting resistance to tyrosine kinase inhibitor chemotherapeutics; (iii) information containing a predetermined control level of markers to be measured with regard to tyrosine kinase inhibitor sensitivity or resistance (e.g., a predetermined control level of a marker of the NTRK1 gene fusion of the present invention that has been correlated with sensitivity to tyrosine kinase inhibitor chemotherapeutics or resistance to tyrosine kinase inhibitor chemotherapeutics).

In another embodiment, a means for detecting the NTRK1 gene fusion markers of the disclosure can generally be any type of reagent that can include, but are not limited to, polynucleotides, hybridization probes, PCR primers, antibodies and antigen binding fragments thereof, peptides, binding partners, aptamers, enzymes, and small molecules. Additional reagents useful for performing an assay using such means for detection can also be included, such as reagents for performing immunohistochemistry, Fluorescent in situ Hybridization (FISH) or a preferred binding assay.

The means for detecting of the assay system of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., DYNABEADS™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a marker described previously herein. Specifically, the means is characterized in that it identifies a specific marker of the cell type being analyzed that positively identifies the cell type. For example, in a lung tumor assay, it is desirable to screen lung cancer cells for the level of the marker expression and/or biological activity. Therefore, the means for detecting a control marker identifies a marker that is characteristic of, for example, a lung cell, so that the cell is distinguished from other cell types, such as a connective tissue or inflammatory cell. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The assay systems and methods of the present invention can be used not only to identify patients that are predicted to be responsive to tyrosine kinase inhibitor chemotherapeutic agents, but also to identify treatments that can improve the responsiveness of cancer cells which are resistant to tyrosine kinase inhibitor chemotherapeutic agents, and to develop adjuvant treatments that enhance the response of cancer patients to tyrosine kinase inhibitor chemotherapeutic agent(s).

The Examples that follow are illustrative of specific embodiments of the invention, and various uses thereof. They are set forth for explanatory purposes only, and are not to be taken as limiting the invention.

### EXAMPLES

### Example 1

This example illustrates the RT-PCR assay performed for detecting the presence of NTRK1-MPRIP fusion gene.

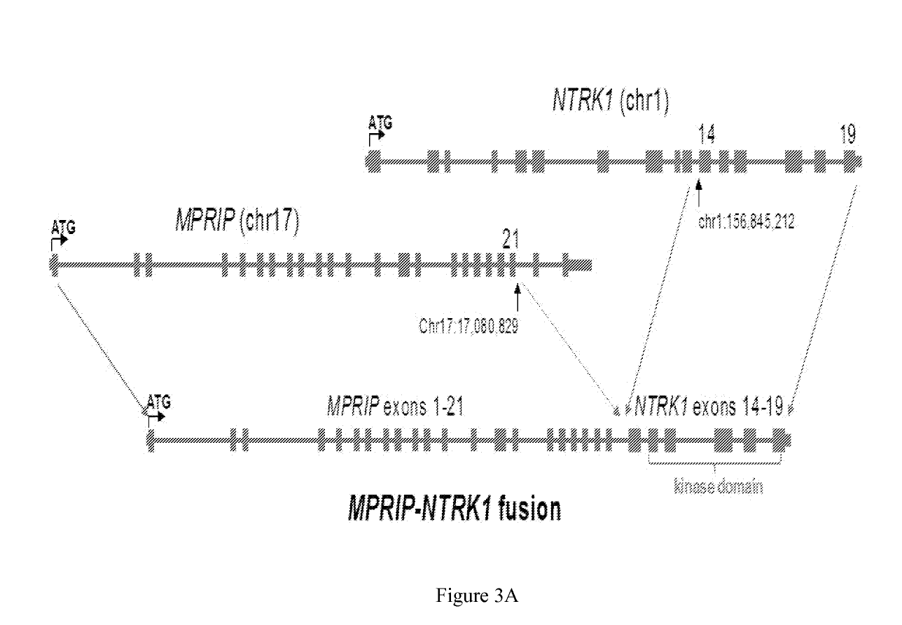

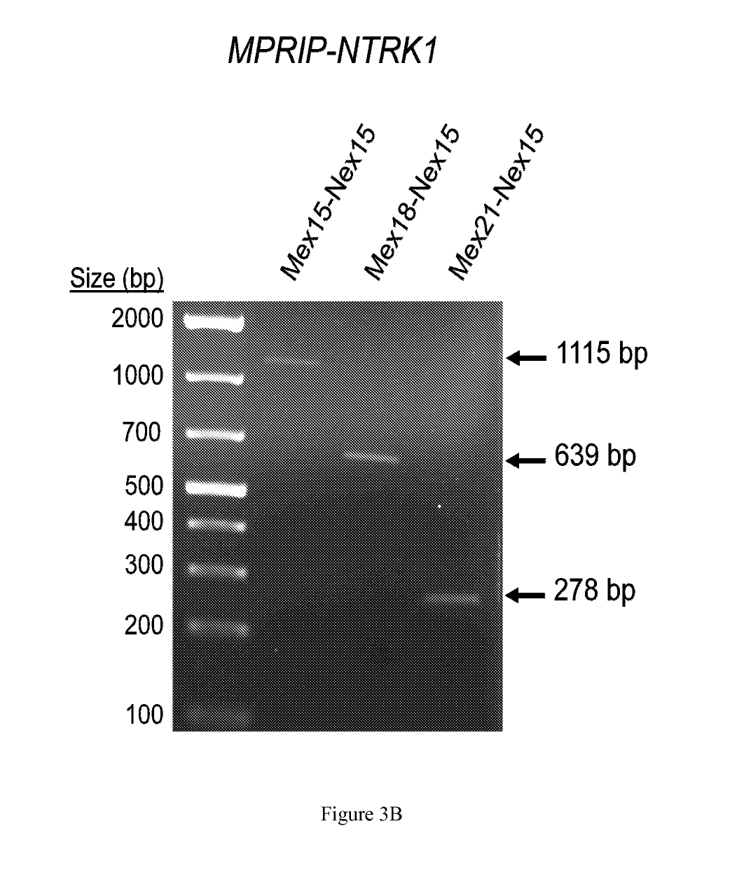

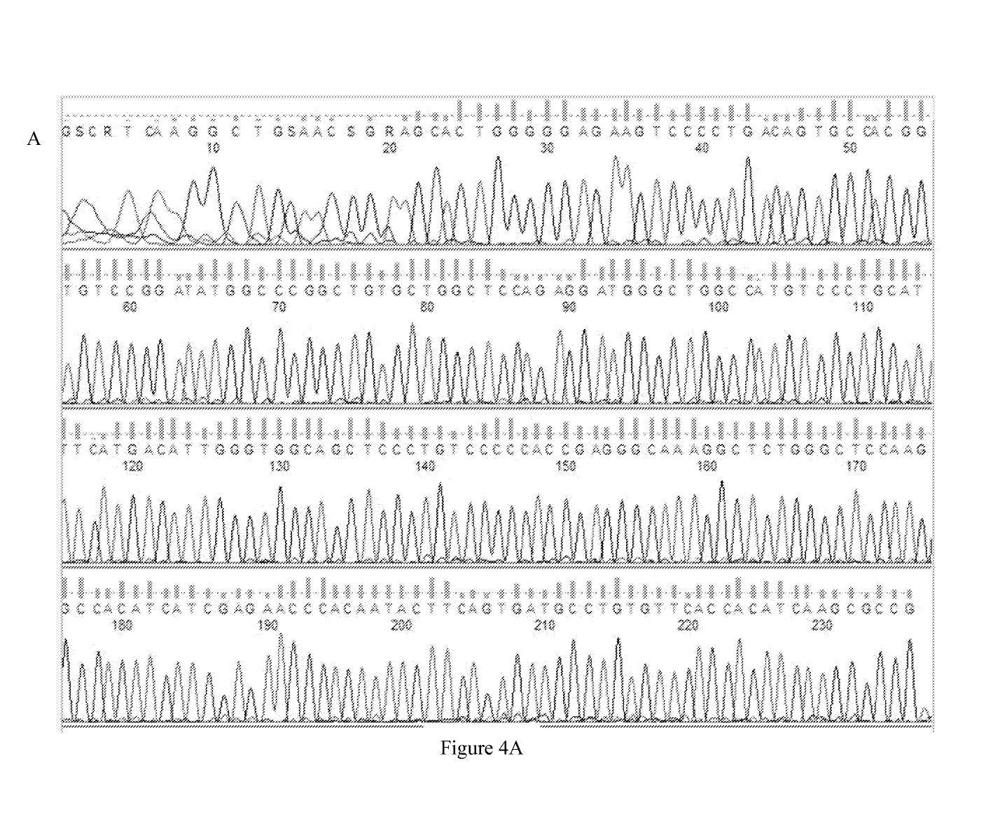

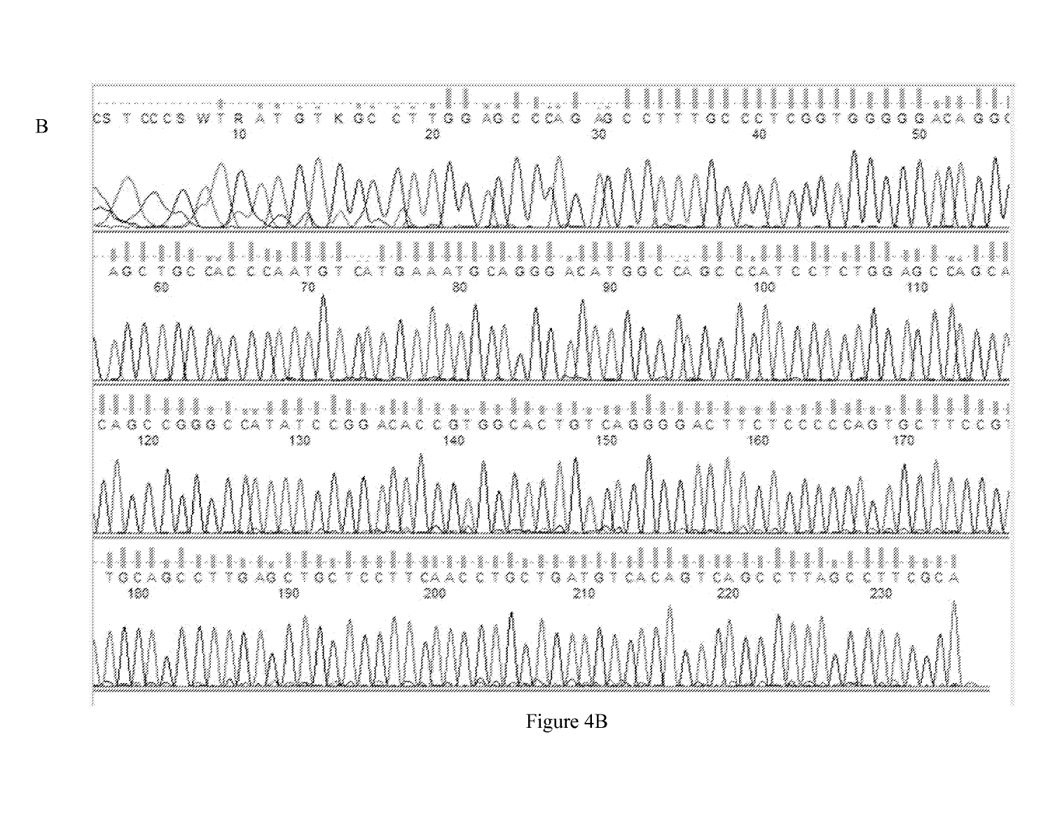

After RNA extraction of a formalin-fixed, paraffin-embedded (FFPE) tumor section sample, a gene specific RT-PCR method was used to identify the NTRK1-MPRIP fusion gene. The resulting RT-PCR reaction generated an approximately 280 bp fragment (see FIG. 3B) that upon sequencing confirmed the presence of a novel in-frame NTRK1-MPRIP fusion gene in which exon 21 of MPRIP is fused to exon 14 of NTRK1. The primer sequences for RT PCR and cloning are as follows:

Primer Name: Primer Sequence (5′ to 3′)
MPRIPStart:
(SEQ ID NO: 2)
ACCATGTCGGCAGCCAAGGAGAACCCGTGC
MPRIP CC1F1:
(SEQ ID NO: 3)
ACACACGAGCTGACCTCTCTGC
MPRIP CC2F1:
(SEQ ID NO: 4)
GTGCCTGGAGAATGCCCATCTG
MPRIP CC3F1:
(SEQ ID NO: 5)
GCGAAGGCTAAGGCTGACTGTG
MPRIP XhoR1:
(SEQ ID NO: 6)
CCATTGCTGCAAACCCTCGCTC
EcoRI MPRIP-Kozak ATG:
(SEQ ID NO: 7)
GAATTCGCCGCCGCGCCGACCATGTCGG
NTRK1Y490R1:
(SEQ ID NO: 8)
CGGCGCTTGATGTGGTGAAC
NTRK1stopR1:
(SEQ ID NO: 9)
TATTCCGGCTAACCACTCCCAG
NTRK1stopR2:
(SEQ ID NO: 10)
CCTAGCCCAGGACATCCAGG
NTRK1 HAstop Not1:
(SEQ ID NO: 11)
CGCGGCCGCTTAAGCGTAGTCTGGGACGTCGTATGGGTAGCCCAGGACA
TCCAGG

RNA extraction from FFPE and Frozen tissues: RNA from FFPE was processed using the RECOVERALL™ Total Nucleic Acid Isolation Kit [Ambion (Austin, Tex.)]. Sections were initially deparaffinized in xylene and washed with 100% ethanol prior to the Protease K digest. After Protease K digest samples were processed for RNA isolation per manufacture instructions.

NTRK1-MPRIP RT-PCR: To identify the fusion breakpoint of MPRIP to NTRK1 from the RNA sample, RT-PCR was carried out using the SUPERSCRIPT™ III First-Strand Synthesis System (Invitrogen) with a NTRK1 primer located in exon 15 of NTRK1 (NTRK Y490R1). For first strand synthesis, RNA, dNTPs and NTRK1 Y490F1 primer were initially denatured at 65° C. for 5 mins and then placed on ice for 2 mins. SUPERSCRIPT™ III reserve transcriptase, RNasin, DTT and reaction buffer was then added to the denatured samples and first strand synthesis was carried out in a PCR machine under the following conditions: 55° C., 10 mins; 50° C. 120 mins; 70° C., 15 mins; 4° C. hold. Following first strand synthesis, the duplexed RNA was removed by an RNase H digest at 37° C. for 20 mins RT-PCR was then performed to amplify NTRK1-MPRIP fusion using the same NTRK1 reverse primer, NTRK1 Y490R1 and a primer to MPRIP located in its 3^{rd} coil-coiled domain (MPRIP CC3F1). PCR conditions for detecting the NTRK1 fusion: initial denaturation at 95° C. for 5 mins; 40 cycles of PCR (95° C. for 30 sec, annealing at 58° C. for 30 sec, and 30 sec extension at 72° C.). PCR products were resolved on a 1.5% agarose gel and the fragments were treated with EXOSAPIT™ (Affymetrix) to remove reaction primers and unincorporated dNTPs. The RT-PCR products were sequenced by the University of Colorado Cancer Center DNA Sequencing and Analysis Core using the same forward and reverse primer in the RT-PCR reaction. The reference sequences used for exon alignment are NCBI Reference Sequences: NM_{—}002529.3 (NTRK1) and NM_{—}015134.3 (MPRIP).

### Example 2

This example illustrates the design of FISH gene fusion probe sets to detect chromosomal rearrangements involving the NTRK1 and MPRIP genes generated by the t(1;17) (q23.1;p11.2) translocation and the break apart probe sets to detect chromosomal rearrangements involving the 5′ and 3′ ends of the NTRK1 gene mapping at 1q23.1. The FISH NTRK1-MPRIP fusion probes and NTRK1 break apart probe sets were developed and validated as described below.

TABLE 1
Detailed features of the reagents used for the NTRK1 beak-apart probe
set and the MPRIP/NTRK1 fusion probe set.

| | Chroro- | | | | | | | Probe | |
|---|---|---|---|---|---|---|---|---|---|
| | some | BAC | Start | End | Length | | | length | Labeled |
| Probe | Band | clones | Point | point | (bp) | dfdf | Primers | (Kb) | color |
| 5′ | 1q23.1 | RP11-891L18 | 156,512,039 | 156,693,389 | 181,351 | Forward | TTCCCAGCTTCTAAGATTCCACCT | 339.4 | Green |
| NTRK1 | | | | | | Reverse | TTTCCCGTGACATTTGGTCCCTTT | | |
| | 1q23.1 | RP11-711O18 | 156,664,877 | 156,851,480 | 186,604 | Forward | TGCATCGAAGTTTGGTTACGGGTT | | |
| | | | | | | Reverse | ACTGGAAATGCTTTGAGGTCAGGA | | |
| 3′ | 1q23.1 | RP11- | 156,854,507 | 156,983,651 | 129,145 | Forward | TGAAAGCCTTCATAGGTGCCTCTT | 331.8 | Red |
| NTRK1 | | 1038N13 | | | | Reverse | TGCAATCAGGGCTGTGAAAGATGT | | |
| | 1q23.1 | RP11- | 156,972,753 | 157,186,313 | 213,561 | Forward | AAACCCAGCCACGAATCTCTTCAA | | |
| | | 1059C21 | | | | Reverse | ACTTGGAAGGAGTGCTGTTGTGTA | | |
| 5′ | 17p11.2 | RP11-125I16 | 16,753,286 | 16,915,525 | 162,240 | Forward | TCTAGTGCAAGGCTCTTCCTCACA | 341.4 | Green |
| MPRIP | | | | | | Reverse | AGACAGCGGAGTGGAGAAGTTGAA | | |
| | 17p11.2 | RP11-796J19 | 16,916,283 | 17,094,721 | 178,439 | Forward | AAGCATGACCTCCAGGGATCTTCA | | |
| | | | | | | Reverse | ATGTGCTTCTGTCCGTGTTCCCTA | | |

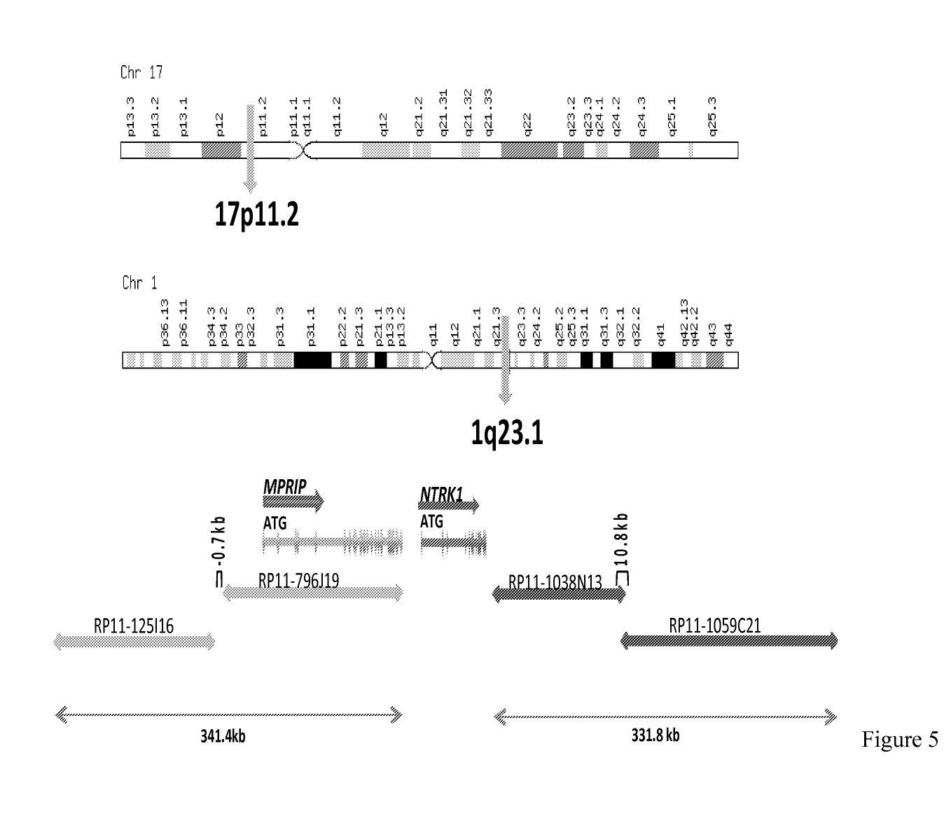

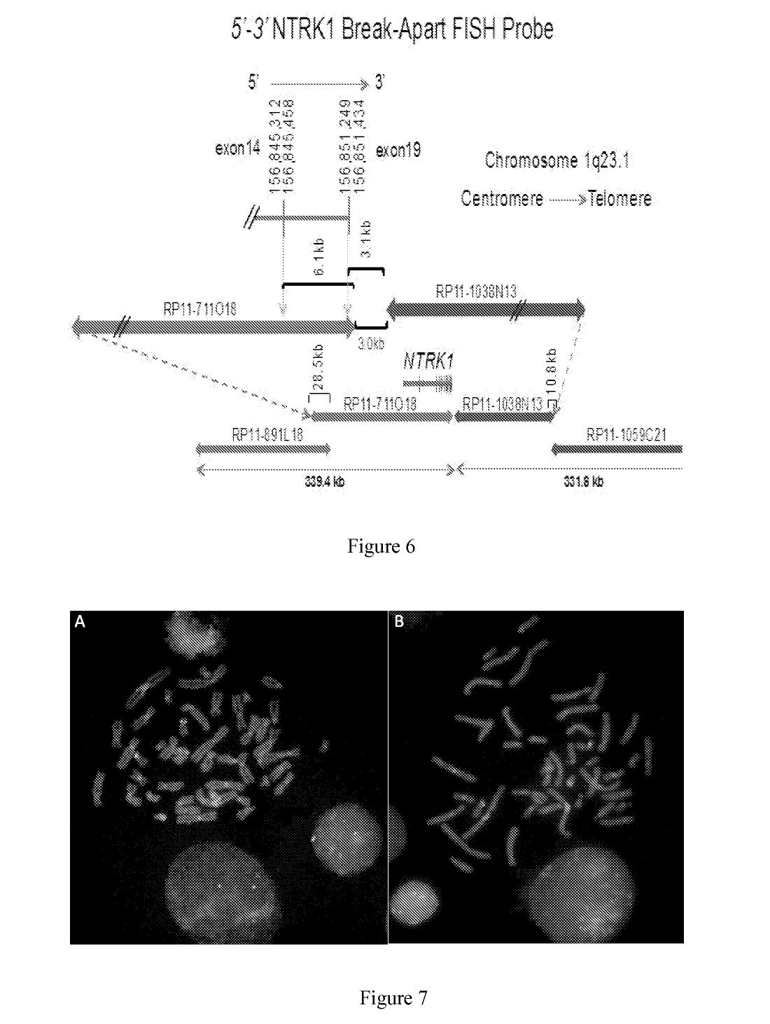

Probe Development: Clone selection, PCR verification, DNA extraction, amplification, and labelling: For the NTRK1-MPRIP fusion probe set, four BAC clones were selected: two for the 3′ NTRK1 probe (labeled with SpectrumRed, SR), recognizing sequences at and downstream (3′) the exon 20 of the NTRK1 gene (RP11-1038N13 and RP11-1059C21), and two for the 5′ MPRIP probe (labeled with SpectrumGreen, SG), recognizing sequences at and upstream (5′) of the exon 29 of the MPRIP gene (RP11-796J19, and RP11-125116). For the Break Apart NTRK1 probe set, two additional BACs (RP11-711018 and RP11-891L18) were selected mapped at and downstream of the 5′ end of the NTRK1 breakpoint. Detailed information for all 6 clones is listed in Table 1 and schematic representation of the fusion and the break-apart probe sets are shown in FIGS. 5 and 6 respectively.

All BAC clones were purchased from BACPAC Resources (CHORI, Oakland Calif.). To verify that the BAC clones encompassed the regions of interest, the specific primers were designed and synthesized by Integrated DNA Technologies. The glycerol stabs were plated on agar plates containing selected antibiotic and 10 single-cell colonies from each BAC clone were selected for PCR verification. Two PCR-validated single colonies per BAC clone had aliquots frozen in glycerol stocks at −86° C.

Mini-cultures of 1 or 2 validated single-cell colonies from each BAC clone were grown in antibiotic-containing LB medium, and genomic DNA was extracted and purified using QIAAMP™ DNA Mini Kit from Qiagen. The purified genomic DNAs from each BAC clone were subject to whole genomic amplification using the REPLI-g Midi Kit from Qiagen.

Amplified human DNA from each BAC clone was labeled in 1 μg aliquots with SpectrumRed conjugated dUTPs (all 3′ NTRK1 probe clones), and SpectrumGreen conjugated dUTPs (all 5′ MPRIP probe clones and all 5′ NTRK1 probe clones), using the Vysis Nick translation kit (Cat# 32-801300), according to the manufacturer's instructions. Each reaction was then treated according to the planned use in validation assays for single clones or combos. Labeled DNAs were co-precipitated with herring sperm DNA as carrier (1:50) and human Cot-1 DNA (1:10) for blocking repetitive sequences and each pellet was diluted in 10 μl of TDENHYB™-2 hybridization buffer from Insitus Biotechnologies for a final concentration of 100 ng/μl.

Definition of Scoring System: Expected signal patterns: In the 5′ MPRIP-3′ NTRK1 FISH fusion probe, the 3′ NTRK1 probe covered 331.8 Kb of the 3′genomic region from the break points of NTRK1 gene in chromosome 1₈23.1. The 5′ MPRIP probe covered 341.4 Kb of the 5′genomic region from the break points of MPRIP gene in chromosome 17. Normal diploid (2N) cells should have two copies of single Red (R), and two copies of single Green (G) signals. In cells carrying a translocation t(1;17)(q23.1;p11.2), the 5′ MPRIP sequence recognized by the (G) probe will be molecularly fused to 3′ NTRK1 sequence recognized by the (R) probe, generating a fused R/G signal. In a normal cell, due to eventual physical co-localization, one copy of R signal and one copy of G signal could be juxtaposed to each other generating a fused R/G signal that mimics the positive signal for the t(1;17). In the 5′ NTRK1-3′ NTRK1 FISH break-apart probe, the 5′ probe covered 339.4 kb upstream the breakpoint and the 3′ NTRK1 probe covered 331.8 Kb downstream the break point of NTRK1 in chromosome 1q23.1. Normal diploid (2 N) cells should have two copies of fused R/G signals and cells carrying a translocation t(1;17)(q23.1;p11.2) should display at least one copy of single G and single R signals.

Analyses of Signal Configuration on Normal Specimens: Typical fluorescent signals are seen as a round and compact spot, named “dot” (see FIG. 30). Atypical signal patterns may be seen in nuclei due to technical variations, probe quality and chromatin stretching. Because the fusion 5′ MPRIP-3′ NTRK1 and the break-apart 5′ NTRK1-3′ NTRK1 probe sets are designed for differentiation between normal and rearranged genes in tumor cells, any atypical configuration of the signals or the relationship (signal pattern) between both probes in a set should be evaluated in detail in normal specimens.

The configuration “Dot” is the typical round and compact signal, as described above (and see FIG. 30). The other categories of split, patchy, and stringy are uncompact signals: split is a divided signal, usually in 2 or 3 fragments; patchy is a diffuse signal with irregular presentation and multiple tiny spots; stringy is an elongated fibrous-like signal. All four of these signal configurations were presented in cell suspension assays. For signal scoring, one dot was counted as 1 signal; a signal with split, patchy or stringy configuration was also counted as 1, even if it consists of 2 or more small spots.

### Example 3

This example illustrates that the FISH fusion probe set for the detection of the MPRIP/NTRK1 gene fusion works efficiently both in cell suspensions and FFPE specimens.

Validation of DNA from single BAC clones and for each of the sets 5′ MPRIP, 5′ NTRK1 and 3′ NTRK1: For validation of the single BAC clones for the Fusion probe set, dual-color FISH assays were performed using combinations of one 3′ NTRK1 probe and one 5′ MPRIP probe [Clones (RP11-1038N13+RP11-125116) and (RP11-1059C21+RP11-796J19)] in the cell line GM09948 (normal karyotype 46,XY). All of the probe mixtures were made up of 100 ng of each of the clones involved and the total volume made up to 4.5 μl with CDENHYB™-2 hybridization buffer for a hybridization area of 113 mm².

FISH assay: All FISH assays in cell suspensions were performed according to standard protocol. Briefly, the slides were treated in a solution of 70% acetic acid for 20-30 sec, incubated in 0.008% pepsin/0.01 M HCL at 37° C. for 3-5 min, fixed in 1% formaldehyde solution for 10 min and dehydrated in graded ethanol series. Probe mix was applied to the selected 12 mm² diameter hybridization areas, which were covered with glass cover slips and sealed with rubber cement. DNA co-denaturation was performed for 8 minutes in an 85° C. dry oven and hybridization was allowed to occur in a moist chamber at 37° C. for 40 hours. Post-hybridization washes were performed with 2× SSC/0.3% NP-40 at 72° C. for 2 min, and 2× SSC for 2 min at room temperature, and dehydrated in graded ethanol series. Chromatin was counterstained with DAPI (0.3μg/ml in Vectashield Mounting Medium, Vector Laboratories).

FISH assays in FFPE specimens were also performed according to standard lab protocols. Specimens were incubated at 56° C. for 4 h, dewaxed in CitriSolv, dehydrated and air-dried, then slides were soaked in 2×SSC at 75° C. for 13≠14 min and digested in 0.6 mg/ml proteinase K at c for 14≠16 min. After dehydration, the fusion probe set 5′ MPRIP-3′ NTRK1 which contained 100ng of each of the 4 BAC clones in 4.5 μl of hybridization buffer was applied to selected 113 mm² hybridization areas and hybridization was allowed to occur for ≠40 h in a humidified chamber at 37° C. Post-hybridization washes were performed as described above for the cell line.

Evaluation of Single BAC Clone Probes in Non-Rearranged Cell Lines

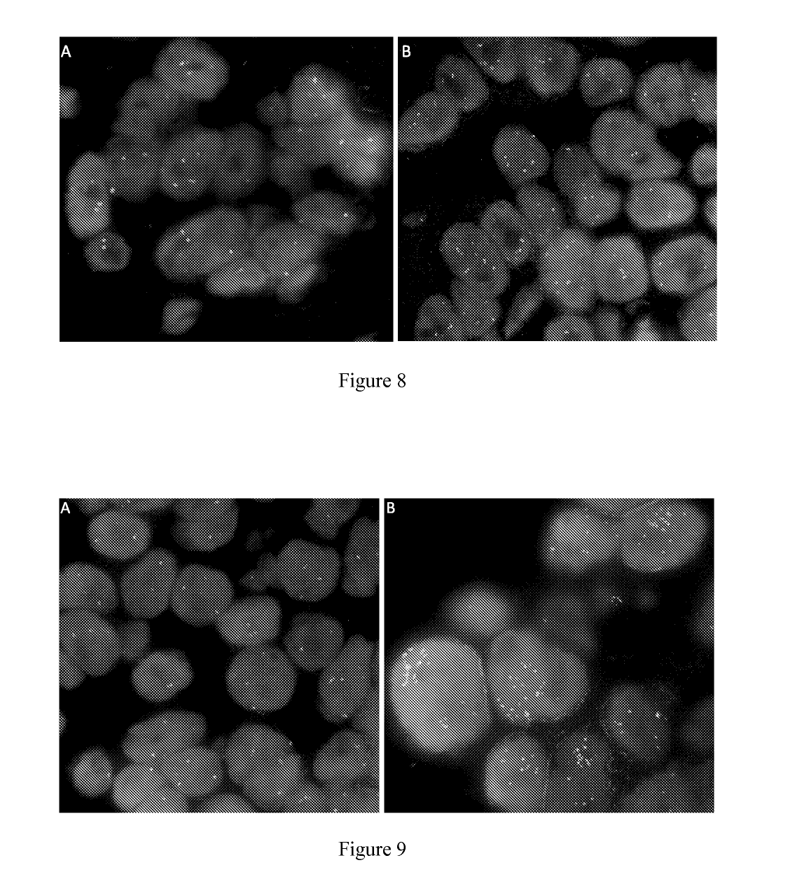

Chromosome Mapping: The quality of the preparations and the intensity of the fluorescence signal were excellent in all slides. Chromosomal mapping was investigated in 25 karyotypically normal metaphase spreads and all of the individual BAC clones mapped correctly: the BAC clones RP11-1038N13 and RP11-1059C21 for 3′ NTRK1 mapped at 1q23.1, the BAC clones RP11-796J19, and RP11-125116 for 5′ MPRIP mapped at 17p11.2 and the BAC clones RP11-711O18/ RP11-891L18 for 5′ NTRK1 also mapped at 1q23.1. The GM09948 cell line had about 99% of the cells with diploid (2N) and 1% of the cells with tetraploid (4N) chromosome content. These cells had, respectively, 2 and 4 copies of each of the clones tested. See FIGS. 7 and 8.

Analysis of Signal Configuration: The signal quality and pattern classification were investigated in 100 diploid interphase nuclei (2N) and the results are summarized in the Table 2.

TABLE 2
Distribution of signals of the MPRIP/NTRKl probe set in
disomic interphase cells according to the configuration.

| Signal | Fre- | 3′NTRKl | 5′ MPRIP |
|---|---|---|---|
| Configuration | quency | (Red) | (G) |
| Dot | N | 118 | 99 |
| | % | 58.7 | 47.8 |
| Split | N | 61 | 52 |
| | % | 30.4 | 25.1 |
| Patchy | N | 12 | 37 |
| | % | 6.0 | 17.9 |
| Stringy | N | 10 | 19 |
| | % | 5.0 | 9.2 |
| Total | N | 201 | 207 |
| | % | 100 | 100 |

Evaluation in FFPE Lung Cancer Sections

Three non-small cell lung cancer specimens were tested with the NTRK1-MPRIP fusion probe set and results of the analyses are shown in Table 3.

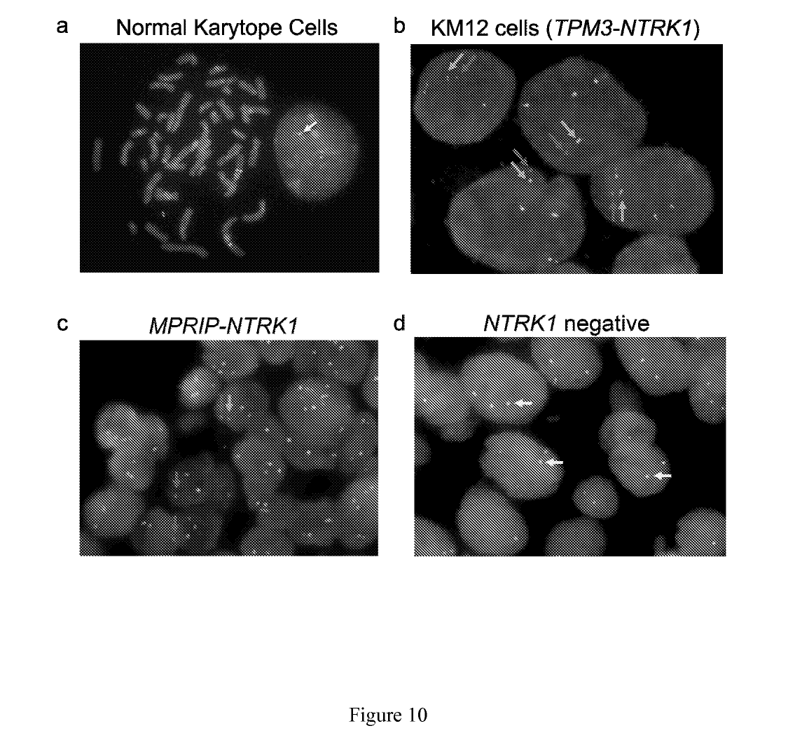

Two specimens were negative for the presence of fusion, with 8% and 9% of cells displaying a typical pattern for positivity. Specimen S-12-047486 (FIG. 8A) had low copy number of each DNA target while specimen S-12-047098 (FIG. 8B) had much higher copy number for both tested targets, albeit higher for NTRK1 than MPRIP. Conversely, the third specimen S-12-6988 B1 showed 88% of cells with typical pattern for NTRK1-MPRIP gene fusion, a clear support for a positive result. Interestingly, as illustrated in FIG. 10, this specimen was heterogeneous, with tumor nuclei ranging in size from medium to very large and harboring, respectively, few copies of each DNA target with one copy of the NTRK1-MPRIP fusion to gene amplification of the NTRK1-MPRIP fusion. These two extreme patterns are illustrated in FIGS. 9A and 9B, respectively.

### Example 4

This example illustrates that the FISH break apart probe set for the detection of the 5′NTRK1/3′NTRK1 gene rearrangement works efficiently both in cell suspensions and FFPE specimens.

The 5′NTRK1/3′NTRK1 Break Apart probe set was validated in the cell line GM09948 (normal karyotype 46,XY) by using the two 3′ NTRK1 probes and the two 5′ NTRK1 probes, (Clones RP11-1038N13/RP11-1059C21+RP11-711O18/ RP11-891L18), because using only one clone of each set will generate split signals, using the same methodology as described above for gene fusion probes. FIGS. 10a-10d show the results. 5′ NTRK1 (green signal) is the proximal end of the gene with respect to the centromere and 3′NTRK1 (red signal) is the distal end of the gene with respect to the centromere. Both probes mapped correctly at 1q23.1.

The 5′NTRK1/3′NTRK1 Break Apart probe set was tested in the GM09948 cell line and specimen S12-6988 B1 as described in Example 3. Analysis was performed on epifluorescence microscope using single interference filters sets for green (FITC), red (Texas red) and blue (DAPI). For each interference filter, monochromatic images were acquired and merged using CytoVision (Leica Microsystems Inc).

The quality of the preparations and the intensity of the fluorescence signals were adequate in all the slides. Chromosomal mapping was investigated in metaphase spreads and the individual BAC clones mapped correctly at 1q23.1 (FIGS. 10a-10d). Evaluation in FFPE lung cancer section was done with specimen S12-6988 B1 (which was shown in Example 3 to have the 5′ MPRIP-3′ NTRK1 rearrangement) and positive patterns were observed (FIG. 11). As shown in FIG. 11 cells show both the ‘positive’ pattern of split red and green and the ‘normal’ pattern of Fused red and green signals.

TABLE 3
Results from hybridization of 3 lung cancer specimens with the 5′MPRIP-3′ NTRK1 Fusion Probe Set

| | | Fused 3′ NTRK1/5′ MPRIP (R/G) | | | | | | Individual 3′ NTRK1 (R) | | | | | |
|---|---|---|---|---|---|---|---|---|---|---|---|---|---|
| | | | | | | % of | | | | | % of | % of | |
| | | | | % of cells | % of cells | cells | % of cells | | | % of cells | cells | cells | % of cells |
| | | | | with 0 | with 1 | with 2 | with ≧3 | | | with 0 | with 1 | with 2 | with ≧3 |
| Specimen ID | FISH assay | Mean | SD | copies | copy | copies | copies | Mean | SD | copies | copy | copies | copies |
| S-12-047486 | FISH12135.1 | 0.07 | 0.26 | 93% | 7% | 0% | 0% | 1.92 | 0.79 | 3% | 21% | 62% | 14% |
| S-12-047098 | FISH12137.2 | 0.11 | 0.37 | 91% | 7% | 2% | 0% | 5.34 | 2.79 | 0% | 0% | 7% | 93% |
| S-12-6988 B1 | FISH12137.1 | 2.54 | 3.53 | 12% | 53% | 9% | 26% | 1.64 | 1.34 | 17% | 35% | 30% | 18% |

| | | | | | | | % cells per category of patterns | | | | | | | |
|---|---|---|---|---|---|---|---|---|---|---|---|---|---|---|
| | Individual 5′ MPRIP (G) | | | | | | | | | | | | Fused + | |
| | | | % of | | % of | % of | | | | | | single | single | % cells |
| | | | cells | % of cells | cells | cells | | only | only | Fused + | Fused + | Red + | Red + | Positive |
| | | | with 0 | with 1 | with 2 | with ≧3 | only | single | single | single | single | single | single | for |
| Specimen ID | Mean | SD | copies | copy | copies | copies | Fused | Red | Green | Red | Green | Green | Green | Rearrangement |
| S-12-047486 | 1.76 | 0.73 | 2% | 32% | 57% | 9% | 0% | 0% | 0% | 2% | 3% | 93% | 2% | 7% |
| S-12-047098 | 2.26 | 1.00 | 0% | 20% | 50% | 30% | 0% | 0% | 0% | 0% | 0% | 91% | 9% | 9% |
| S-12-6988 B1 | 1.48 | 1.10 | 12% | 49% | 26% | 13% | 8% | 0% | 0% | 4% | 9% | 12% | 67% | 88% |

### Example 5

This example demonstrates that activated TRKA (the protein product of the NTRK1 gene) can be inhibited by several small molecule tyrosine kinase inhibitors.

Gene fusion events lead to activation of the kinase domain encoded in the 3′ end of the fusion gene by leading to increased expression via the promoter of the 5′ gene in the fusion and often by inducing dimerization through domains (such as coiled-coil domains) encoded within the 5′ gene fusion partner. This enhanced dimerization leads to constitutive activation of the kinase domain, in this case the kinase domain of TRKA.

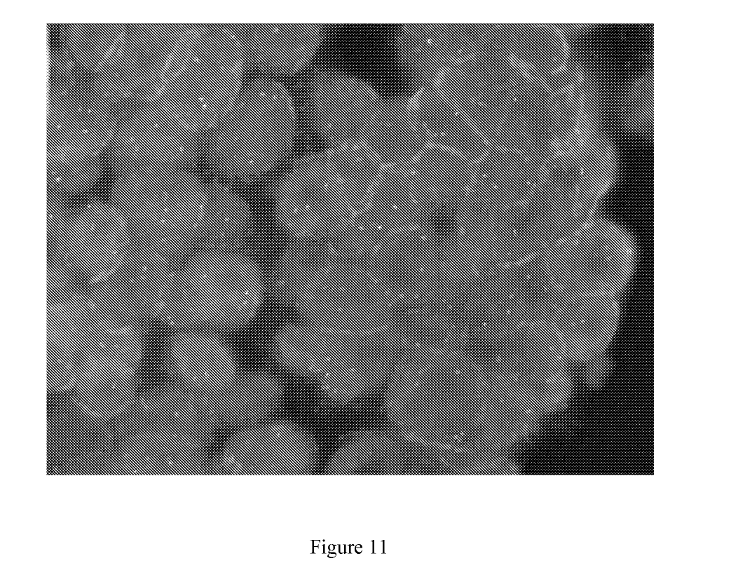

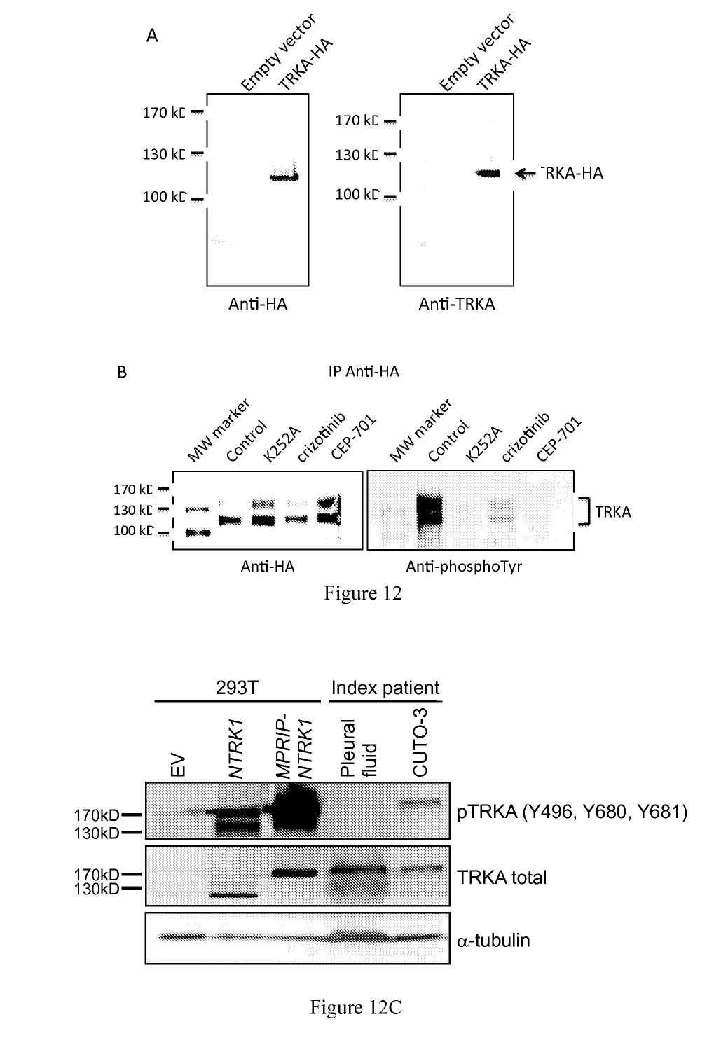

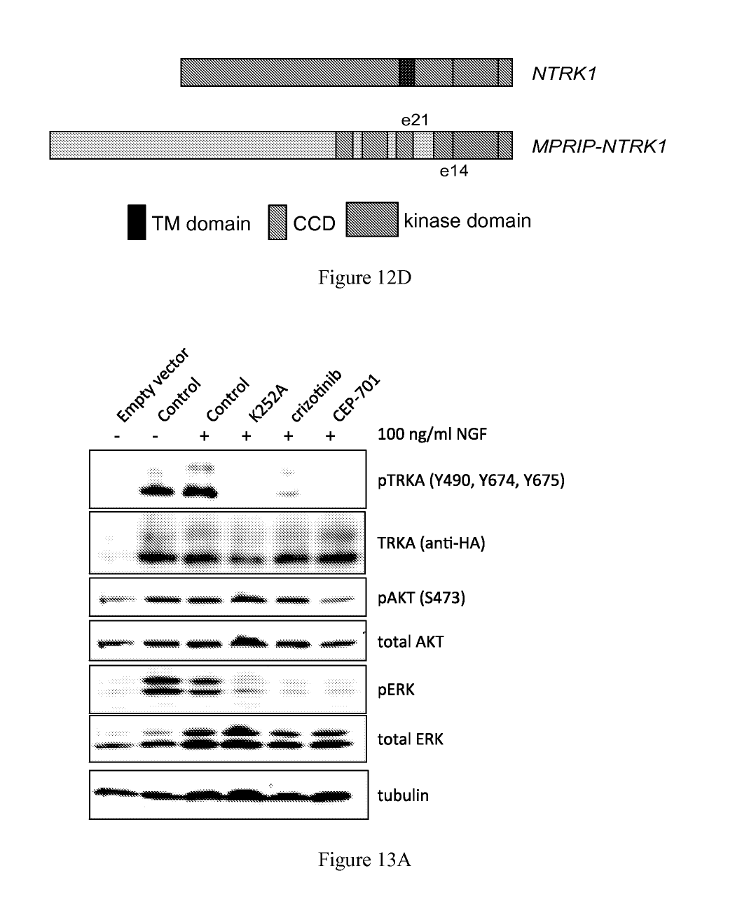

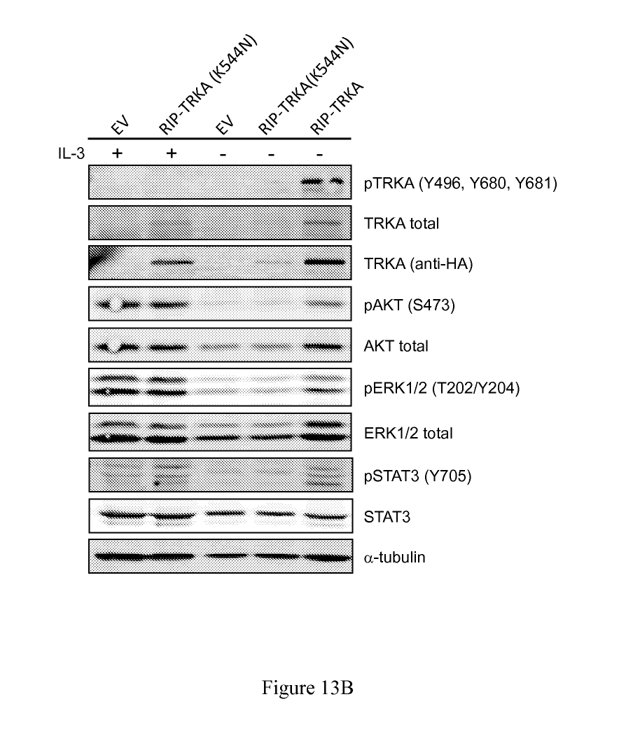

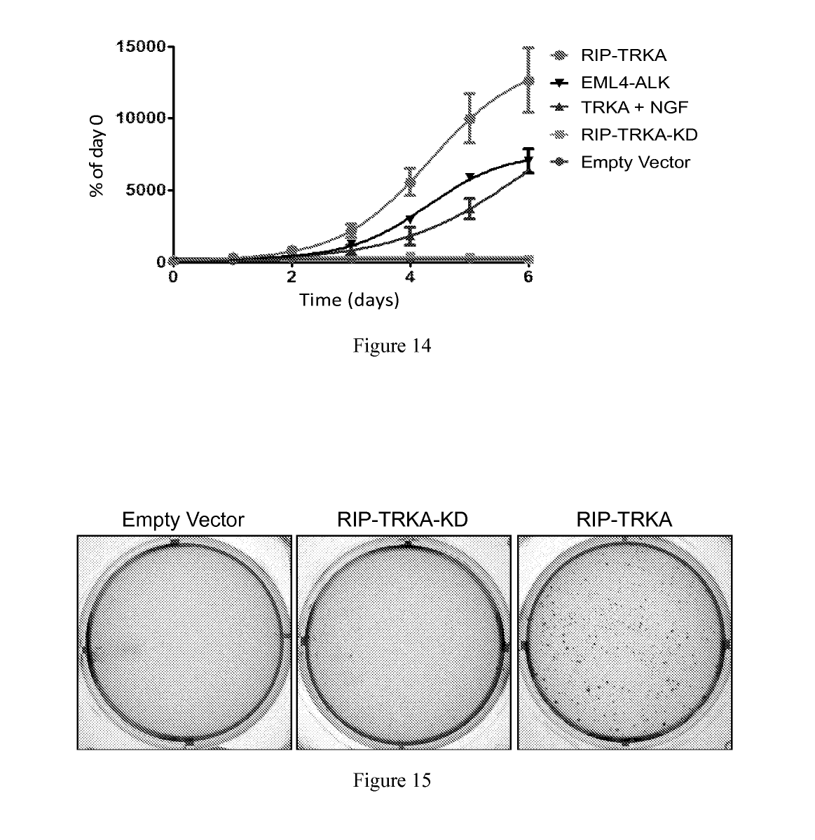

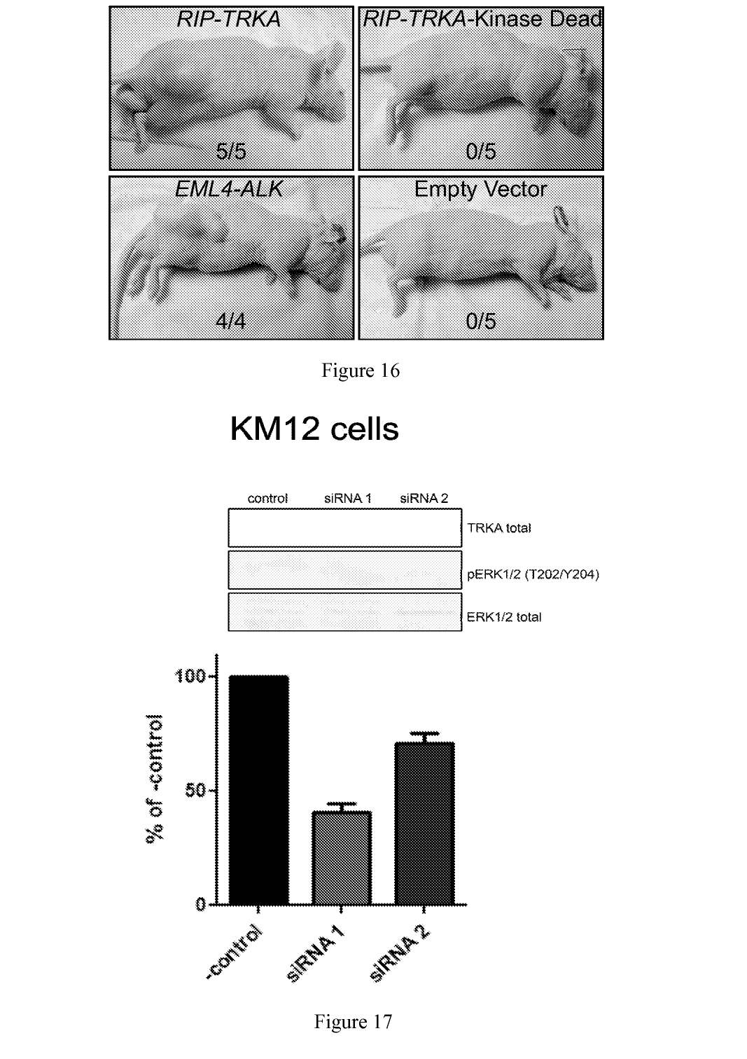

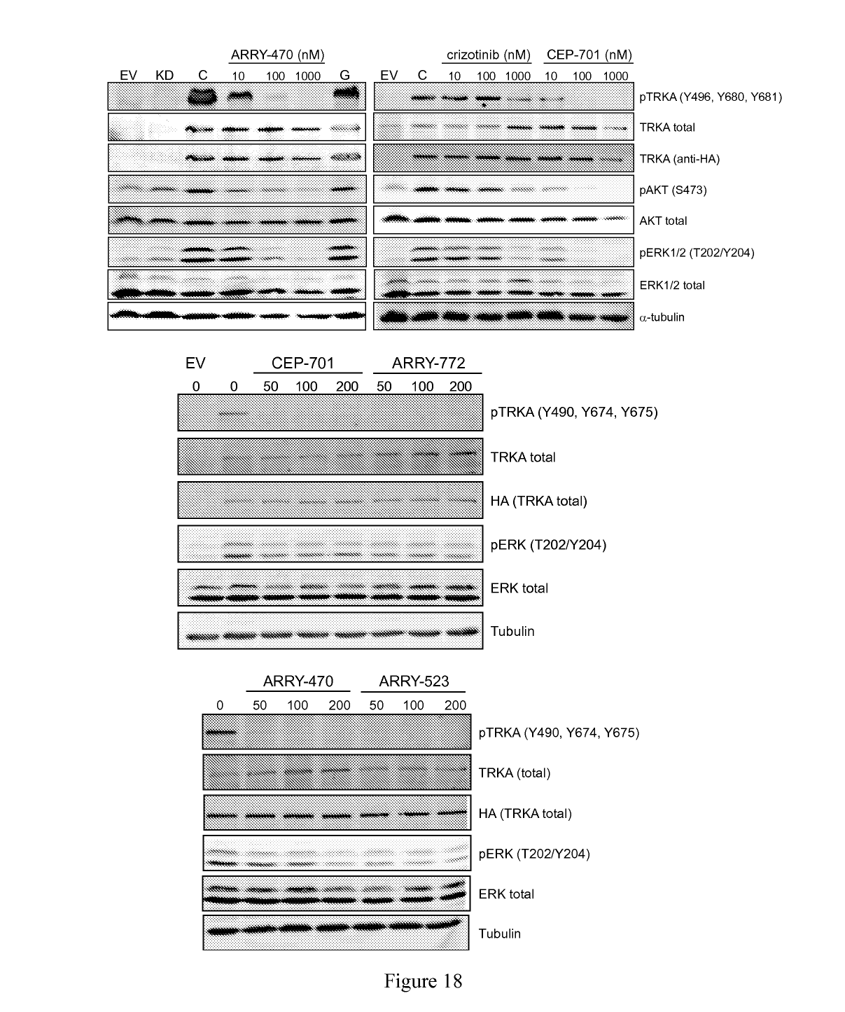

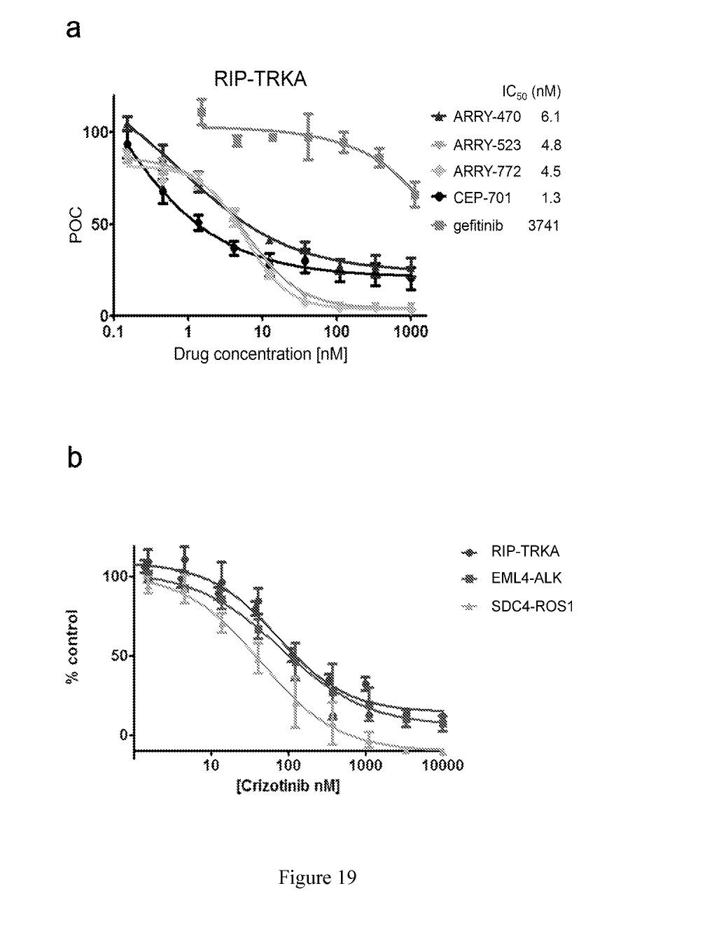

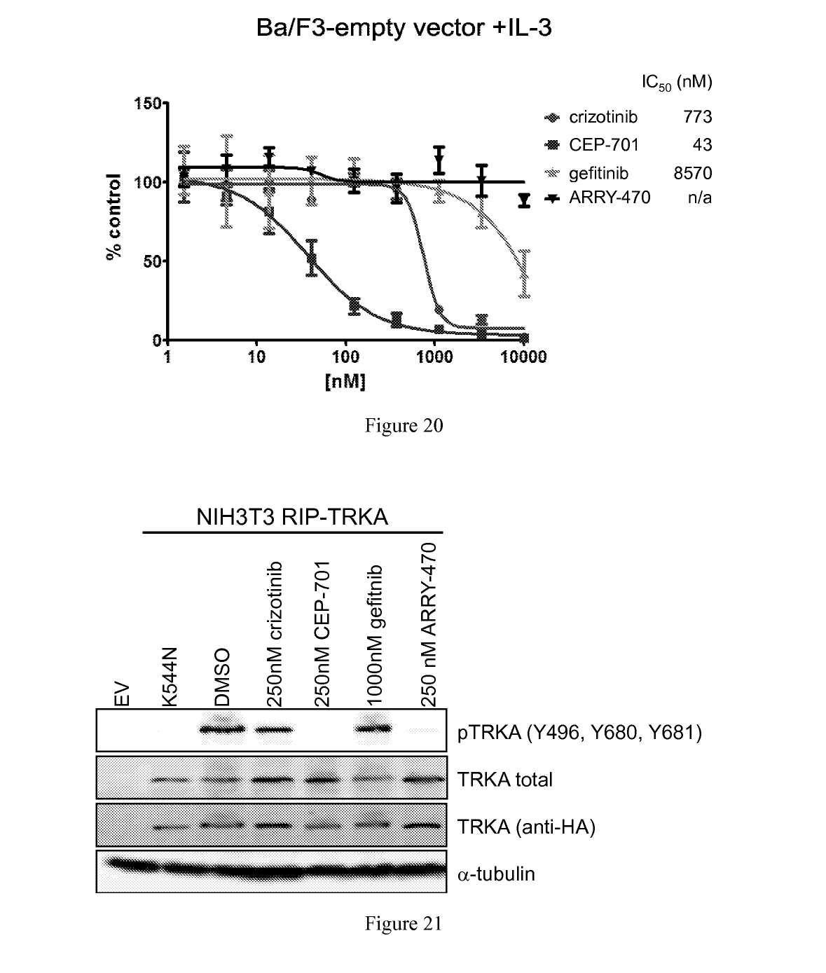

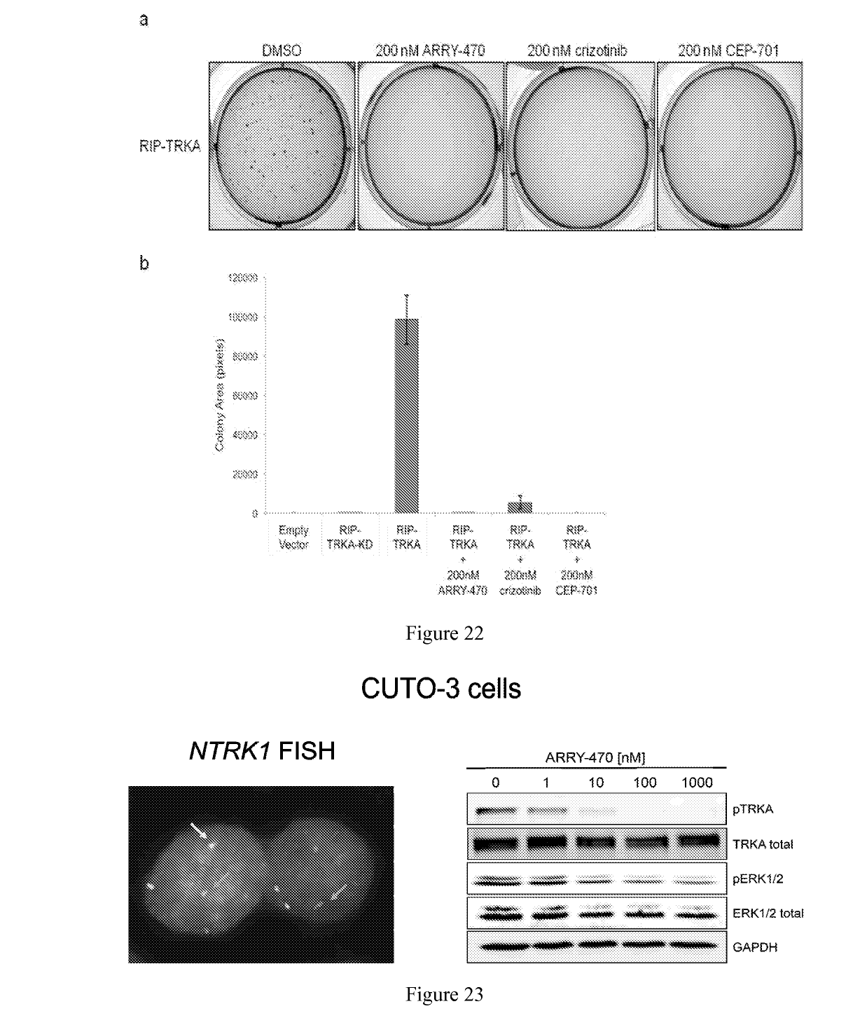

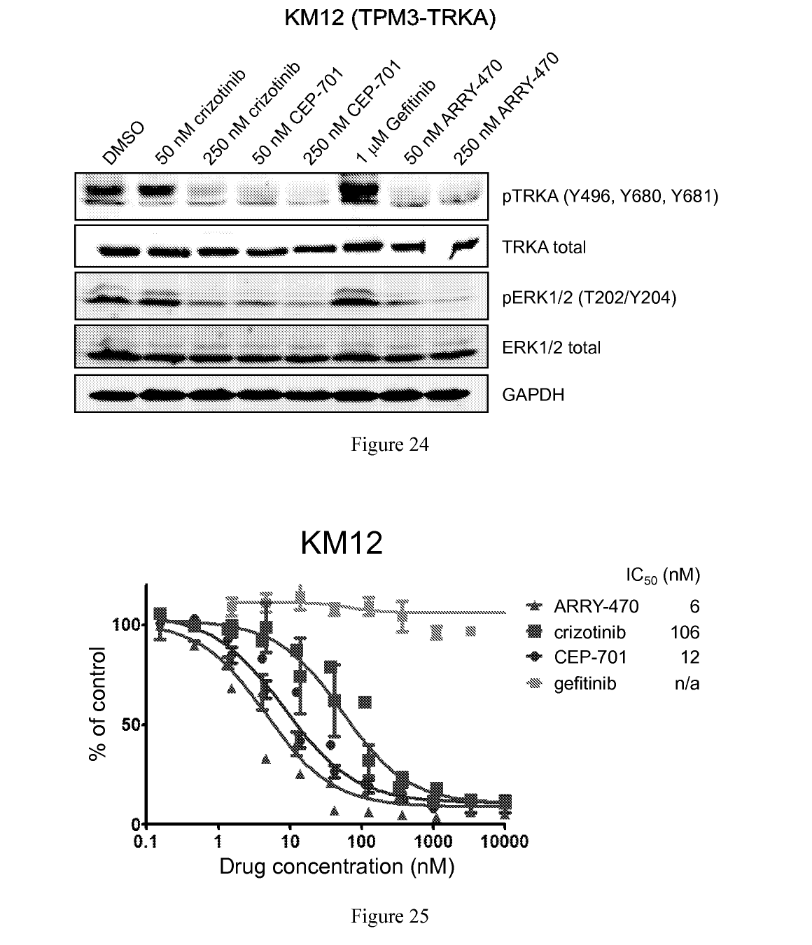

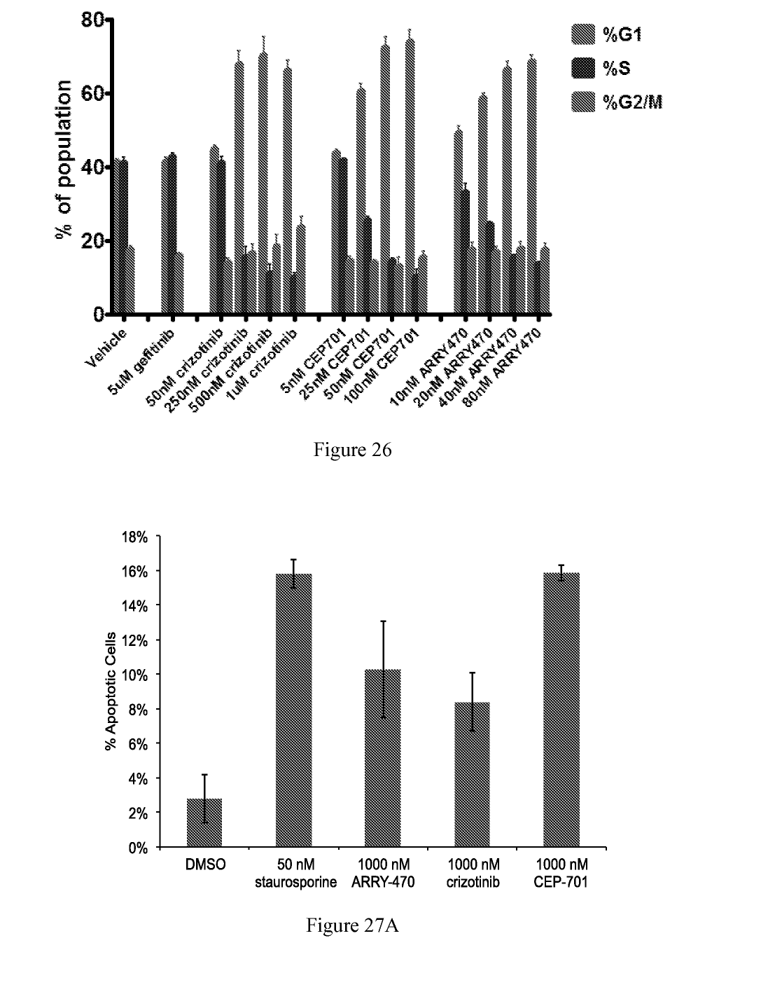

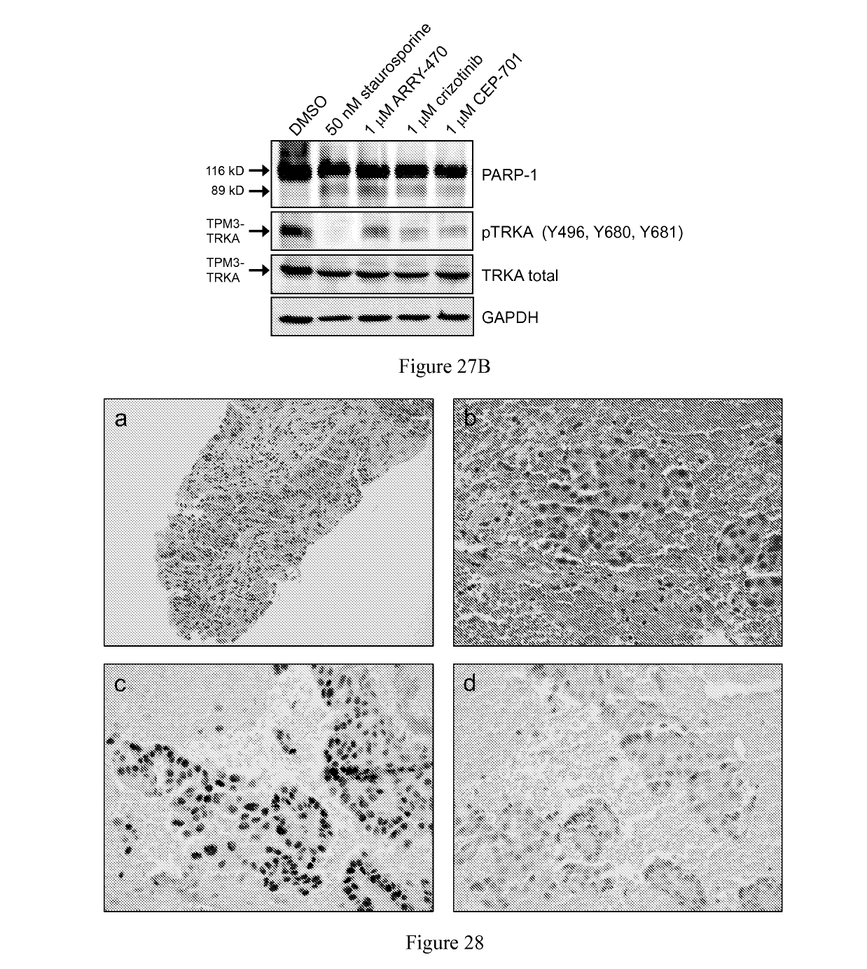

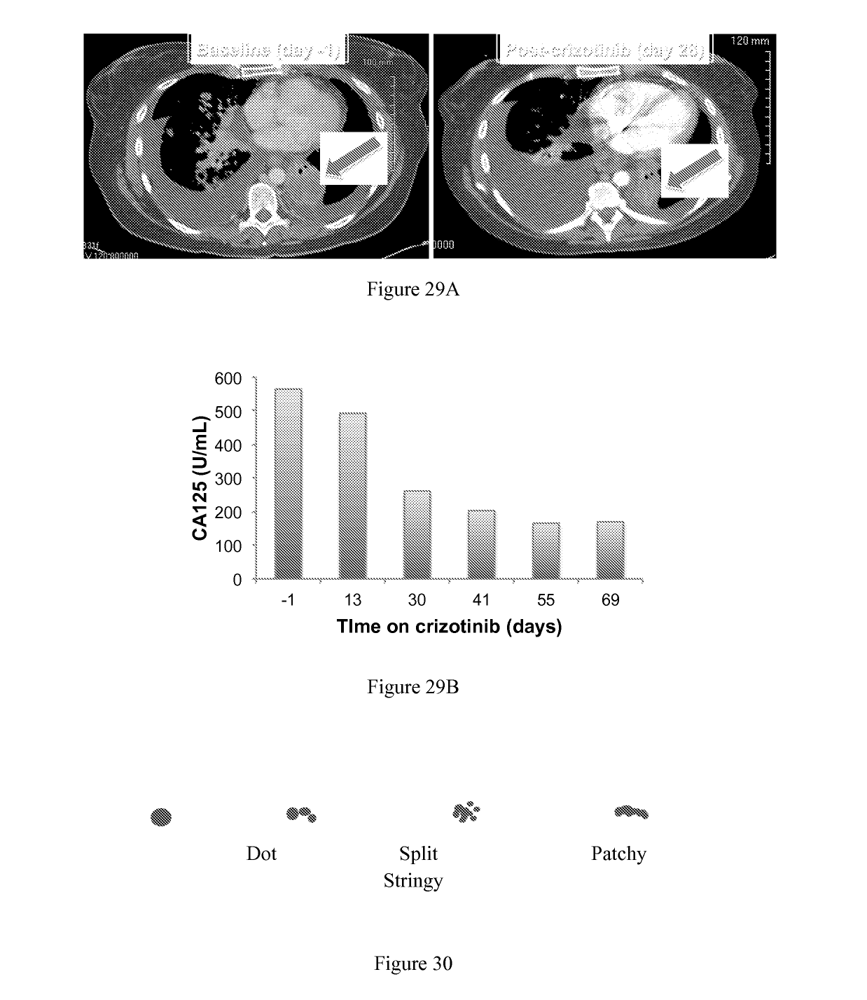

To determine whether small molecular tyrosine kinase inhibitors inhibit TRKA, the inventors expressed full length TRKA in 293T cells (FIGS. 12 and 13). The NTRK1 gene was cloned into pCDH-MCS1-EF1-puro with the addition of a hemagglutinin (HA) tag at the 3′ (C-terminal) end. This vector was transiently transfected into 293T cells which demonstrated expression of TRKA compared to the empty vector control using an antibody to both HA (Cell Signaling, C29F4) and TRKA (Santa Cruz, C-118) in immunoblot analysis (FIG. 12A). FIG. 12A shows expression of an approximate 115-120kD protein detected by an HA-specific antibody (left, Cell Signaling) and a TRKA-specific antibody (right, Santa Cruz, SC-118). Immunoprecipation of TRKA using the HA-specific antibody followed by immunobloting with an anti-phosphotyrosine antibody (Millipore, 4G10) demonstrated significant phosphorylation of TRKA in the DMSO-treated (control) sample (FIG. 12B). Phosphorylation of TRKA was significantly reduced after treatment of cells with 1 μM of either K252A, crizotinib, or CEP-701 (all purchased from Selleck Chemical) for 5 hours, indicating inhibition of TRKA activity. FIG. 12B shows immunoprecipation using an HA-specific antibody (Cell Signaling) followed by immunoblot using the same antibody (left) or a phosphotyrosine specific antibody (right) following treatment with 1 μM of the indicated inhibitors or DMSO (control) for 5 hours. FIG. 12C shows that expression of NTRK1-MPRIP yields a chimeric protein that is autophosphorylated Immunoblot analysis of 293T cells transiently transfected with empty vector (EV), full length NTRK1 cDNA, NTRK1-MPRIP cDNA compared to tumor cells from a frozen pleural fluid sample or early passage cells in culture (CUTO-3) from the index patient with the NTRK1-MPRIP fusion gene.

Expression of TRKA was detected in cell lysates and this protein was activated in the absence or presence of its ligand, nerve growth factor (NGF), as detected by immunoblot analysis using antibodies to phosphotyrosines at TRKA amino acid positions 490, 674 and 675 (Cell Signaling) (FIG. 13A). Further evidence of TRKA activity was seen by increased phosphorylation of downstream signaling pathways such as AKT (Cell Signaling, S473) or ERK (Cell Signaling). Treatment of cells with 1 μM of either K252A or crizotinib led to decreased phosphorylation of TRKA and ERK, whereas CEP-701 also inhibited phosphorylation of AKT in addition to TRKA and ERK. FIG. 13 shows SDS-PAGE of 293T cell lysates with expression of TRKA-HA or empty vector in the presence or absence of NGF (10 minutes) and the presence or absence of the indicated tyrosine kinase inhibitors at 1 μM for 5 hours. Membranes were probed with antibodies to TRKA phosphotyrosine 490, 674, and 675 (Cell Signaling), total TRKA (anti-HA, Cell Signaling), AKT phosphoserine 473 (Cell Signaling), total AKT (Cell Signaling), phosphorylated ERK p42/44 (Cell Signaling), total ERK p42/44 (Cell Signaling), and tubulin (Santa Cruz, SC-8035).

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended Claims. All publications and references are herein expressly incorporated by reference in their entirety.

1. Ferlay J, Shin H R, Bray F, Forman D, Mathers C, Parkin D M. Estimates of worldwide burden of cancer in 2008: GLOBOCAN 2008. Int J Cancer 2010;127:2893-917.

2. Wells S A, Jr., Gosnell J E, Gagel R F, et al. Vandetanib for the treatment of patients with locally advanced or metastatic hereditary medullary thyroid cancer. J Clin Oncol 2010;28:767-72.

3. Mok TS, Wu YL, Thongprasert S, et al. Gefitinib or carboplatin-paclitaxel in pulmonary adenocarcinoma. N Engl J Med 2009;361:947-57.

4. Kwak E L, Bang Y J, Camidge D R, et al. Anaplastic lymphoma kinase inhibition in non-small-cell lung cancer. N Engl J Med 2010;363:1693-703.

5. Soda M, Choi Y L, Enomoto M, et al. Identification of the transforming EML4-ALK fusion gene in non-small-cell lung cancer. Nature 2007;448:561-6.

6. Rikova K, Guo A, Zeng Q, et al. Global survey of phosphotyrosine signaling identifies oncogenic kinases in lung cancer. Cell 2007;131:1190-203.

7. Takeuchi K, Choi Y L, Togashi Y, et al. KIF5B-ALK, a novel fusion oncokinase identified by an immunohistochemistry-based diagnostic system for ALK-positive lung cancer. Clin Cancer Res 2009;15:3143-9.

8. Birchmeier C, Sharma S, Wigler M. Expression and rearrangement of the ROS1 gene in human glioblastoma cells. Proc Natl Acad Sci U S A 1987;84:9270-4.

9. Charest A, Lane K, McMahon K, et al. Fusion of FIG to the receptor tyrosine kinase ROS in a glioblastoma with an interstitial del(6)(q21q21). Genes Chromosomes Cancer 2003;37:58-71.

10. Bergethon K, Shaw A T, Ou S H, et al. ROS1 rearrangements define a unique molecular class of lung cancers. J Clin Oncol 2012;30:863-70.

11. Rimkunas V M, Crosby K, Kelly M, et al. Analysis of Receptor Tyrosine Kinase ROS1 Positive Tumors in Non-small Cell Lung Cancer: Identification of a FIG-ROS1 Fusion. Clin Cancer Res 2012.

12. Takeuchi K, Soda M, Togashi Y, et al. RET, ROS1 and ALK fusions in lung cancer. Nat Med 2012;18:378-81.

13. Davies K D, Le A T, Theodoro M F, et al. Identifying and Targeting ROS1 Gene Fusions in Non-Small Cell Lung Cancer. Clin Can Res;In press.

14. Nikiforov Y E, Nikiforova M N. Molecular genetics and diagnosis of thyroid cancer. Nat Rev Endocrinol 2011;7:569-80.

15. Herbst R S, Heymach J V, O'Reilly M S, Onn A, Ryan A J. Vandetanib (ZD6474): an orally available receptor tyrosine kinase inhibitor that selectively targets pathways critical for tumor growth and angiogenesis. Expert Opin Investig Drugs 2007;16:239-49.

16. Doebele R C, Pilling A B, Aisner D L, et al. Mechanisms of resistance to crizotinib in patients with ALK gene rearranged non-small cell lung cancer. Clin Cancer Res 2012;18:1472-82.

17. Davies K D, Le A T, Theodoro M F, et al. Targeting ROS1 Gene Fusions in Non-Small Cell Lung Cancer. Clin Can Res Submitted.

18. Camidge D R, Kono S A, Flacco A, et al. Optimizing the detection of lung cancer patients harboring anaplastic lymphoma kinase (ALK) gene rearrangements potentially suitable for ALK inhibitor treatment. Clin Cancer Res 2010;16:5581-90.

19. Camidge D R, Theodoro M, Maxson D A, et al. Correlations between the percentage of tumor cells showing an ALK (anaplastic lymphoma kinase) gene rearrangement, ALK signal copy number, and response to crizotinib therapy in ALK fluorescence in situ hybridization-positive nonsmall cell lung cancer. Cancer 2012.

20. Pierotti M A, Bongarzone I, Borello M G, Greco A, Pilotti S, Sozzi G. Cytogenetics and molecular genetics of carcinomas arising from thyroid epithelial follicular cells. Genes Chromosomes Cancer 1996;16:1-14.

21. Hondermarck H. Neurotrophins and their receptors in breast cancer. Cytokine Growth Factor Rev 2012.

22. Knezevich S R, McFadden D E, Tao W, Lim J F, Sorensen P H. A novel ETV6-NTRK3 gene fusion in congenital fibrosarcoma. Nat Genet 1998;18:184-7.

23. Tognon C, Knezevich S R, Huntsman D, et al. Expression of the ETV6-NTRK3 gene fusion as a primary event in human secretory breast carcinoma. Cancer Cell 2002;2:367-76.

24. Kralik J M, Kranewitter W, Boesmueller H, et al. Characterization of a newly identified ETV6-NTRK3 fusion transcript in acute myeloid leukemia. Diagn Pathol 2011;6:19.

25. Cui J J, Tran-Dube M, Shen H, et al. Structure based drug design of crizotinib (PF-02341066), a potent and selective dual inhibitor of mesenchymal-epithelial transition factor (c-MET) kinase and anaplastic lymphoma kinase (ALK). J Med Chem 2011;54:6342-63.

26. O'Hare T, Shakespeare W C, Zhu X, et al. AP24534, a pan-BCR-ABL inhibitor for chronic myeloid leukemia, potently inhibits the T315I mutant and overcomes mutation-based resistance. Cancer Cell 2009;16:401-12.

27. Karaman M W, Herrgard S, Treiber D K, et al. A quantitative analysis of kinase inhibitor selectivity. Nat Biotechnol 2008;26:127-32.

28. George D J, Dionne C A, Jani J, et al. Sustained in vivo regression of Dunning H rat prostate cancers treated with combinations of androgen ablation and Trk tyrosine kinase inhibitors, CEP-751 (KT-6587) or CEP-701 (KT-5555). Cancer Res 1999;59:2395-401.

29. Wang T, Yu D, Lamb M L. Trk kinase inhibitors as new treatments for cancer and pain. Expert Opin Ther Pat 2009;19:305-19.

What is claimed is:

1. A method to select a cancer patient who is predicted to respond to the administration of a chemotherapeutic regimen comprising:
detecting in a sample of tumor cells from the patient the presence or absence of a NTRK1-MPRIP gene fusion;
selecting the patient as predicted to respond to the administration of a chemotherapeutic regimen comprising an agent selected from the group consisting of a tyrosine kinase inhibitor, an HSP90 inhibitor, an inhibitor of tyrosine kinase downstream signalling cascade, and combinations thereof if a NTRK1-MPRIP gene fusion is detected in the sample of tumor cells; or
selecting the patient as predicted to not respond to the administration of a chemotherapeutic regimen comprising an agent selected from the group consisting of a tyrosine kinase inhibitor, an HSP90 inhibitor, an inhibitor of tyrosine kinase downstream signalling cascade, and combinations thereof if a NTRK1-MPRIP gene fusion is not detected in the sample of tumor cells.

2. The method of claim 1, wherein the detection comprises detecting a level of the NTRK1-MPRIP gene fusion present in the sample of tumor cells and, comparing the level to a standard level or reference range.

3. The method of claim 2, wherein the standard level or reference range is determined according to a statistical procedure for risk prediction.

4. The method of claim 1, wherein the presence of the NTRK1-MPRIP gene fusion is determined by detecting the presence of a polynucleotide.

5. The method of claim 4, wherein the presence of the NTRK1-MPRIP gene fusion is determined by Fluorescent In Situ Hybridization (FISH).

6. The method of claim 1, wherein the presence of the NTRK1-MPRIP gene fusion is determined by detecting the presence of a polypeptide.

7. The method of claim 6, wherein the method comprises detecting the presence of the polypeptide using at least one of an antibody, an antibody derivative, and an antibody fragment, that specifically binds to the polypeptide or a fragment thereof.

8. The method of claim 1, wherein the detecting of the NTRK1-MPRIP gene fusion comprises:
obtaining RNA from the sample of tumor cells;
generating cDNA from the RNA;
amplifying the cDNA with PCR primers specific for the NTRK1-MPRIP gene fusion selected from the group consisting of:
(SEQ ID NO: 2)
ACCATGTCGGCAGCCAAGGAGAACCCGTGC;
(SEQ ID NO: 3)
ACACACGAGCTGACCTCTCTGC;
(SEQ ID NO: 4)
TGCCTGGAGAATGCCCATCTG;
(SEQ ID NO: 5)
GCGAAGGCTAAGGCTGACTGTG;
(SEQ ID NO: 6)
CCATTGCTGCAAACCCTCGCTC;
(SEQ ID NO: 7)
GAATTCGCCGCCGCGCCGACCATGTCGG;
(SEQ ID NO: 8)
CGGCGCTTGATGTGGTGAAC;
(SEQ ID NO: 9)
TATTCCGGCTAACCACTCCCAG;
(SEQ ID NO: 10)
CCTAGCCCAGGACATCCAGG;
and
(SEQ ID NO: 11)
CGCGGCCGCTTAAGCGTAGTCTGGGACGTCGTATGGGTAGCCCAGGAC
ATCCAGG;
determining the presence or absence of the NTRK1-MPRIP gene fusion in the amplified cDNA.

9. The method of claim 1, wherein the patient is a human.

10. (canceled)

11. The method of claim 1, further comprising:
comparing the level of the NTRK1-MPRIP gene fusion in the sample of tumor cells to a level of the NTRK1-MPRIP gene fusion in a second patient predicted to not respond to the administration of a chemotherapeutic regimen including one or more kinase inhibitor(s), and,
selecting the patient as being predicted to respond to the administration of a chemotherapeutic regimen including one or more kinase inhibitor(s), if the expression level of the NTRK1-MPRIP gene fusion in the sample of tumor cells is greater than the level of expression of the NTRK1-MPRIP gene fusion in the second patient, or, selecting the patient as being predicted to not respond to the administration of a chemotherapeutic regimen including one or more kinase inhibitor(s), if the level of the NTRK1-MPRIP gene fusion in the sample of tumor cells is less than or equal to the level of the NTRK1-MPRIP gene fusion in the second patient.

12. The method of claim 1, wherein the tyrosine kinase inhibitor is selected from the group consisting of crizotinib (PF-02340166), ponatinib (AP24534), dovitinib (TK-258), rebastinib (DCC-2036), CEP-701, AZD-7451, ARRY-470, ARRY-523, ARRY-772 and combinations thereof.

13. The method of claim 1, wherein the tyrosine kinase inhibitor is a TrkA inhibitor.

14. An assay system for predicting response or outcome of a cancer patient to the administration of tyrosine kinase inhibitor therapy comprising a means to detect at least one of:
a) the presence of a NTRK1-MPRIP gene fusion;
b) the level of expression of a gene transcript encoded by a NTRK1-MPRIP gene fusion;
c) the presence of a protein encoded by a NTRK1-MPRIP gene fusion;
d) the level of a protein encoded by a NTRK1-MPRIP gene fusion; and,
e) the activity of a protein encoded by a NTRK1-MPRIP gene fusion.

15. The assay system of claim 14, wherein the means to detect comprises nucleic acid probes comprising at least 10 to 50 contiguous nucleic acids of NTRK1 gene, or complementary nucleic acid sequences thereof.

16. The assay system of claim 14, wherein the means to detect comprises binding ligands that specifically detect polypeptides encoded by a NTRK1-MPRIP gene fusion.

17. The assay system of claim 14, comprising an assay surface comprising a chip, array, or fluidity card.

18. The assay system of claim 14, further comprising: a control selected from the group consisting of:
information containing a predetermined control level of a gene transcript encoded by a NTRK1-MPRIP gene fusion that has been correlated with response to the administration of a chemotherapeutic regimen including one or more tyrosine kinase inhibitor(s); and
information containing a predetermined control level of a gene transcript encoded by a NTRK1-MPRIP gene fusion that has been correlated with a lack of response to the administration of a chemotherapeutic regimen including one or more tyrosine kinase inhibitor(s).

19-27. (canceled)

28. An isolated nucleic acid selected from the group consisting of:
(SEQ ID NO: 2)
ACCATGTCGGCAGCCAAGGAGAACCCGTGC;
(SEQ ID NO: 3)
ACACACGAGCTGACCTCTCTGC;
(SEQ ID NO: 4)
TGCCTGGAGAATGCCCATCTG;
(SEQ ID NO: 5)
GCGAAGGCTAAGGCTGACTGTG;
(SEQ ID NO: 6)
CCATTGCTGCAAACCCTCGCTC;
(SEQ ID NO: 7)
GAATTCGCCGCCGCGCCGACCATGTCGG;
(SEQ ID NO: 8)
CGGCGCTTGATGTGGTGAAC;
(SEQ ID NO: 9)
TATTCCGGCTAACCACTCCCAG;
(SEQ ID NO: 10)
CCTAGCCCAGGACATCCAGG;
and,
(SEQ ID NO: 11)
CGCGGCCGCTTAAGCGTAGTCTGGGACGTCGTATGGGTAGCCCAGGA/
CATCCAGG.

29. A method of treating a cancer patient comprising administering to a patient diagnosed with a cancer and having a NTRK1-MPRIP gene fusion detected in a sample of tumor cells from the patient, a therapeutically effective amount of at least one anti-cancer compound selected from the group consisting of geldanamycin, herbimycin, 17-AAG, PU24FC1, STA-9090, IPI-504, and AUY-922, crizotinib, ponatinib, dovitinib, rebastinib, CEP-701, AZD-7451, ARRY-470, ARRY-523, and ARRY-772.


SEQUENCE LISTING (11 sequences)

SEQ ID NO: 1 - DNA, 4300 bp, Homo sapiens
gcggccgcgctgagcccctagcccgccgggagcgccaggccggccaggcctgcgccgccgccgccgccgccgtcgccgccgcgccgaccatgtcggcagccaaggagaacccgtgcaggaaattccaggccaacatcttcaacaagagcaagtgtcagaactgcttcaagccccgcgagtcgcatctgctcaacgacgaggacctgacgcaggcaaaacccatttatggcggttggctgctcctggctccagatgggaccgactttgacaacccagtgcaccggtctcggaaatggcagcgacggttcttcatcctttacgagcacggcctcttgcgctacgccctggatgagatgcccacgacccttcctcagggcaccatcaacatgaaccagtgcacagatgtggtggatggggagggccgcacgggccagaagttctccctgtgtattctgacgcctgagaaggagcatttcatccgggcggagaccaaggagatcgtcagtgggtggctggagatgctcatggtctatccccggaccaacaagcagaatcagaagaagaaacggaaagtggagccccccacaccacaggagcctgggcctgccaaggtggctgttaccagcagcagcagcagcagcagcagcagcagcagcatccccagtgctgagaaagtccccaccaccaagtccacactctggcaggaagaaatgaggaccaaggaccagccagatggcagcagcctgagtccagctcagagtcccagccagagccagcctcctgctgccagctccctgcgggaacctgggctagagagcaaagaagaggagagcgccatgagtagcgaccgcatggactgtggccgcaaagtccgggtggagagcggctacttctctctggagaagaccaaacaggacttgaaggctgaagaacagcagctgcccccgccgctctcccctcccagccccagcacccccaaccacaggaggtcccaggtgattgaaaagtttgaggccttggacattgagaaggcagagcacatggagaccaatgcagtggggccctcaccatccagcgacacacgccagggccgcagcgagaagagggcgttccctaggaagcgggacttcaccaatgaagcccccccagctcctctcccagacgcctcggcttcccccctgtctccacaccgaagagccaagtcactggacaggaggtccacggagccctccgtgacgcccgacctgctgaatttcaagaaaggctggctgactaagcagtatgaggacggccagtggaagaaacactggtttgtcctcgccgatcaaagcctgagatactacagggattcagtggctgaggaggcagccgacttggatggagaaattgacttgtccgcatgttacgatgtcacagagtatccagttcagagaaactatggcttccagatacatacaaaggagggcgagtttaccctgtcggccatgacatctgggattcggcggaactggatccagaccatcatgaagcacgtgcacccgaccactgccccggatgtgaccagctcgttgccagaggaaaaaaacaagagcagctgctcttttgagacctgcccgaggcctactgagaagcaagaggcagagctgggggagccggaccctgagcagaagaggagccgcgcacgggagcggaggcgagagggccgctccaagacctttgactgggctgagttccgtcccatccagcaggccctggctcaggagcgggtgggcggcgtggggcctgctgacacccacgagcccctgcgccctgaggcggagcctggggagctggagcgggagcgtgcacggaggcgggaggagcgccgcaagcgcttcgggatgctcgacgccacagacgggccaggcactgaggatgcagccctgcgcatggaggtggaccggagcccagggctgcctatgagcgacctcaaaacgcataacgtccacgtggagattgagcagcggtggcatcaggtggagaccacacctctccgggaagagaagcaggtgcccatcgcccccgtccacctgtcttctgaagatgggggtgaccggctctccacacacgagctgacctctctgctcgagaaggagctggagcagagccagaaggaggcctcagaccttctggagcagaaccggctcctgcaggaccagctgagggtggccctgggccgggagcagagcgcccgtgagggctacgtgctgcaggccacgtgcgagcgagggtttgcagcaatggaagaaacgcaccagaagaagattgaagatctccagaggcagcaccagcgggagctagagaaacttcgagaagagaaagaccgcctcctagccgaggagacagcggccaccatctcagccatcgaagccatgaagaacgcccaccgggaggaaatggagcgggagctggagaagagccagcggtcccagatcagcagcgtcaactcggatgttgaggccctgcggcgccagtacctggaggagctgcagtcggtgcagcgggaactggaggtcctctcggagcagtactcgcagaagtgcctggagaatgcccatctggcccaggcgctggaggccgagcggcaggccctgcggcagtgccagcgtgagaaccaggagctcaatgcccacaaccaggagctgaacaaccgcctggctgcagagatcacacggttgcggacgctgctgactggggacggcggtggggaggccactgggtcaccccttgcacagggcaaggatgcctatgaactagaggtcttattgcgggtaaaggaatcggaaatacagtacctgaaacaggagattagctccctcaaggatgagctgcagacggcactgcgggacaagaagtacgcaagtgacaagtacaaagacatctacacagagctcagcatcgcgaaggctaaggctgactgtgacatcagcaggttgaaggagcagctcaaggctgcaacggaagcactgggggagaagtcccctgacagtgccacggtgtccggatatggcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctggcaaggtcccgtcatagcaattatatttattatcccttgaaaaaaaa

SEQ ID NO: 2 - DNA, 30 bp, Artificial Sequence
  Synthetic primer
accatgtcggcagccaaggagaacccgtgc

SEQ ID NO: 3 - DNA, 22 bp, Artificial Sequence
  Synthetic primer
acacacgagctgacctctctgc

SEQ ID NO: 4 - DNA, 22 bp, Artificial Sequence
  Synthetic primer
gtgcctggagaatgcccatctg

SEQ ID NO: 5 - DNA, 22 bp, Artificial Sequence
  Synthetic primer
gcgaaggctaaggctgactgtg

SEQ ID NO: 6 - DNA, 22 bp, Artificial Sequence
  Synthetic primer
ccattgctgcaaaccctcgctc

SEQ ID NO: 7 - DNA, 28 bp, Artificial Sequence
  Synthetic primer
gaattcgccgccgcgccgaccatgtcgg

SEQ ID NO: 8 - DNA, 20 bp, Artificial Sequence
  Synthetic primer
cggcgcttgatgtggtgaac

SEQ ID NO: 9 - DNA, 22 bp, Artificial Sequence
  Synthetic primer
tattccggctaaccactcccag

SEQ ID NO: 10 - DNA, 20 bp, Artificial Sequence
  Synthetic primer
cctagcccaggacatccagg

SEQ ID NO: 11 - DNA, 55 bp, Artificial Sequence
  Synthetic primer
cgcggccgcttaagcgtagtctgggacgtcgtatgggtagcccaggacatccagg